SEQUENCE LISTING (4 sequences)

SEQ ID NO: 1 - DNA, 20 bp, Oryza sativa
tacatctccggcgtttacac

SEQ ID NO: 2 - DNA, 20 bp, Oryza sativa
ccaaggttgaaacaggaagc

SEQ ID NO: 3 - DNA, 20 bp, Oryza sativa
gattctcgtcgtaatcaaga

SEQ ID NO: 4 - DNA, 20 bp, Oryza sativa
atccacggttaagataaatg

(19) United States
(12) Patent Application Publication
(10) Pub. No.: US 2018/0070548 A1
(43) Pub. Date: Mar. 15, 2018

(54) OXYFLUORFEN RESISTANT RICE LINES

(71) Applicant: CALIFORNIA COOPERATIVE RICE RESEARCH FOUNDATION, INC., Biggs, CA (US)

(72) Inventor: KENT SCHEIDEL MCKENZIE, OROVILLE, CA (US)

(21) Appl. No.: 15/675,183

(22) Filed: Aug. 11, 2017

Related U.S. Application Data
(60) Provisional application No. 62/395,039, filed on Sep. 15, 2016.

(51) Int. Cl. A01H 5/10; A01H 4/00; A01H 1/02; C12N 15/82; C07K 14/325; C07K 14/415

(52) U.S. Cl. CPC A01H 5/10; A01H 4/00; A01H 1/02; C12N 15/8274; C12N 15/8278; C12N 15/8275; C12N 15/8245; C12N 15/8286; C07K 14/325; C07K 14/415; C12N 15/8279; C12N 15/8247; C12N 15/8277

(57) ABSTRACT

The present invention is directed toward rice lines containing mutant allele ROXY that exhibit non-transgenic resistance to the herbicide oxyfluorfen. The invention relates to the seeds, plants and plant parts of rice plants containing mutant allele ROXY and to methods for producing a rice plant by crossing rice plants containing mutant allele ROXY with itself or another rice cultivar. The invention further relates to methods for producing a rice plant containing mutant allele ROXY containing in its genetic material one or more transgenes and to the transgenic rice plants and plant parts produced by those methods. This invention also relates to rice cultivars or breeding cultivars and plant parts derived from rice plants containing mutant allele ROXY, to methods for producing other rice cultivars, lines or plant parts derived from rice plants containing mutant allele ROXY and to the rice plants, varieties, and their parts derived from the use of those methods. The invention further relates to transferring mutant allele ROXY to different genetic backgrounds.

### CROSS REFERENCE TO RELATED APPLICATIONS

This application claims the benefit of priority from U.S. provisional patent application Ser. No. 62/395,039 filed on Sep. 15, 2016, which is incorporated herein by reference in its entirety.

### BACKGROUND OF THE INVENTION

The present invention relates to new rice plants, seeds, varieties and hybrids having a mutant allele designated ROXY, which confers resistance to the herbicide oxyfluorfen. The present invention relates to mutant rice plant lines that have high levels of resistance to the herbicide oxyfluorfen (Goal® 2XL and GoalTender®). The mutant lines provide the opportunity to use an established effective crop herbicide that is too damaging to conventional rice plants by using rice varieties that have this non-transgenic oxyfluorfen resistance trait. In addition, the present invention is also directed to transferring the ROXY allele to plants in the same species lacking the allele, and is useful for producing novel plants and varieties of rice having resistance to oxyfluorfen. All publications cited in this application are herein incorporated by reference.

Rice is an ancient agricultural crop and is today one of the principal food crops of the world. There are two cultivated species of rice: Oryza sativa L., the Asian rice, and O. glaberrima Steud., the African rice. O. sativa L. constitutes virtually all of the world's cultivated rice and is the species grown in the United States. Three major rice producing regions exist in the United States: the Mississippi Delta (Arkansas, Miss., northeast Louisiana, southeast Missouri), the Gulf Coast (southwest Louisiana, southeast Texas), and the Central Valleys of California.

Rice is a semi-aquatic crop that benefits from flooded soil conditions during part or all of the growing season. In the United States, rice is grown on flooded soils to optimize grain yields. Heavy clay soils or silt loam soils with hard pan layers about 30 cm below the surface are typical rice-producing soils because they minimize water losses from soil percolation. Rice production in the United States can be broadly categorized as either dry-seeded or water-seeded. In the dry-seeded system, rice is sown into a well-prepared seed bed with a grain drill or by broadcasting the seed and incorporating it with a disk or harrow. Moisture for seed germination is from irrigation or rainfall. Another method of planting by the dry-seeded system is to broadcast the seed by airplane into a flooded field, then promptly drain the water from the field. For the dry-seeded system, when the plants have reached sufficient size (four- to five-leaf stage), a shallow permanent flood of water 5 to 16 cm deep is applied to the field for the remainder of the crop season. It is a desirable to have rice varieties that grow quickly as seedlings to compete with weeds and hasten the application of a permanent flood that suppresses the growth of grassy weeds. Residual grass herbicides are also used to prevent the emergence of new weeds.

In the water-seeded system, rice seed is soaked for 12 to 36 hours to initiate germination, and the seed is broadcast by airplane into a flooded field. This is the predominant rice production system in California. Some herbicides are applied pre-emergence (applied to soil before flooding) and some applied at the date of seeding or early in the first few weeks of rice seedling growth. Controlling weeds at germination or when they are young reduces competition with the rice seedling, hastening the rice plant growth and canopy closure that helps suppress weeds. Weeds are also much easier to control when in the early stages of growth. Pre-emergence application have advantages to the grower in that they can be accomplished at the final stage of seedbed preparation by ground as opposed to an aerial application when the field is flooded.

Aerial application of pesticides is subject to regulation and restriction due to potential drift or movement, and some rice herbicide materials cannot be applied by air in California. In some cases the herbicides are not liquid sprays but granules that disperse in the flooded paddy to control the weeds. This is of great advantage to prevent drift or movement of the herbicide from the target field. Maintaining a permanent flood supports the control of grassy weeds, especially in combination with selective grass herbicides. The aquatic weeds (e.g. sedges and rushes), however are favored by the permanent flood. The water-seeded system lends itself to the water infrastructure, delivery to the fields, and management in California.

The rice seedlings emerge through a shallow flood, or the water may be drained from the field for a short period of time to enhance seedling establishment. Lowering the water improves stand establishment by providing additional oxygen that enhances root growth and better anchoring the seedling against uprooting by wind and wave action. It also exposes weeds for contact herbicide applications. However, this promotes grassy weed growth, nitrogen loss, and increased water consumption and management. Herbicide use on rice can injure the rice plant and reduce plant growth, shorten the height, delay maturity and possibly reduce yield. This may be the result of high or cool temperatures. Draining the field or lowering the water is used to lessen herbicide injury; however, this may not be possible because of water hold periods required for an herbicide, and water management and use efficiency. In recent years, due to environmental regulations, appearance of herbicide resistant weeds and phasing out of older herbicides, weed control in commercial rice production in California has become a primary production issue for growers.

A shallow flood is maintained until the rice approaches maturity. For both the dry-seeded and water-seeded production systems, the fields are drained when the crop is mature, and the rice is harvested 2 to 3 weeks later with large combines. In rice breeding programs, breeders try to employ the production systems predominant in their respective region. Thus, a drill-seeded breeding nursery is used by breeders in a region where rice is drill-seeded and a water-seeded nursery is used in regions where water-seeding is important.

Rice in the United States is classified into three primary market types by grain size, shape, and chemical composition of the endosperm: long-grain, medium grain and short-grain. Typical U. S. long-grain cultivars cook dry and fluffy when steamed or boiled, whereas medium- and short-grain cultivars cook moist and sticky. Long-grain cultivars have been traditionally grown in the southern states and generally receive higher market prices.

Although specific breeding objectives vary somewhat in the different regions, increasing yield is a primary objective in all programs. Grain yield of rice is determined by the number of panicles per unit area, the number of fertile florets per panicle, and grain weight per floret. Increases in any or all of these yield components may provide a mechanism to obtain higher yields. Heritable variation exists for all of these components, and breeders may directly or indirectly select for increases in any of them.

Grain weight is a very important yield component in rice. Genetic control of grain weight is typically quantitatively inherited. Large kernel size is often a desirable quality feature.

There are some important traits in rice that are controlled by single genes or genes of major effect and are simply inherited. One of the most notable is the semidwarf habit that is controlled by the sd gene. This gene has been used extensively to produce high yielding short stature rice varieties and has been the subject of extensive research, including the actual sequencing of the gene and the various alleles.

Herbicide tolerant rice mutants have been used to develop rice varieties that are resistant to an herbicide, that will control several weed species, and even the weedy red rice that is the same genus and species as cultivated rice Oryza sativa L. see “Clearfield-AHAS” T. P. Croughan U.S. Patent Pub. No. 2015/02161126 A1, Aug. 6, 2015; “Provisia ACCase” from BASF, Mankin et al. U.S. Patent Pub. No. 2014/0045686 A1, Feb. 13, 2014; “RiceTec ACCase” Hinga et al. U.S. Patent Pub. No. 2015/0038331 A1, Feb. 15, 2015.

Rice, Oryza sativa L., is an important and valuable field crop. Thus, a continuing goal of rice plant breeders is to develop stable, high yielding rice cultivars that are agronomically sound. The reasons for this goal are obviously to maximize the amount of grain produced on the land used and to supply food for both animals and humans. To accomplish this goal, the rice breeder must select and develop rice plants that have the traits that result in superior cultivars.

The foregoing examples of the related art and limitations related therewith are intended to be illustrative and not exclusive. Other limitations of the related art will become apparent to those of skill in the art upon a reading of the specification.

### SUMMARY OF THE INVENTION

The following embodiments and aspects thereof are described in conjunction with systems, tools and methods which are meant to be exemplary and illustrative, not limiting in scope. In various embodiments, one or more of the above described problems have been reduced or eliminated, while other embodiments are directed to other improvements.

In one aspect of the invention, there is provided a novel mutant allele designated ROXY that confers a high level of resistance to the herbicide oxyfluorfen. The present invention relates to plants, seeds, and other plant parts such as pollen and ovules containing mutant allele ROXY. The present invention further relates to methods for producing rice lines with a high level of resistance to oxyfluorfen by crossing rice plants containing mutant allele ROXY with itself or with another rice line, and the creation of variants by mutagenesis or transformation of rice plants containing mutant allele ROXY. The invention further relates to rice plants produced by said methods.

According to the invention, there are provided novel rice lines, botanically known as Oryza sativa L. that exhibit non-transgenic resistance to the herbicide oxyfluorfen. This invention thus relates to the seeds of rice lines that exhibit oxyfluorfen resistance, to the plants or part(s) thereof of rice lines that exhibit oxyfluorfen resistance, to the plants or part(s) thereof having all of the phenotypic and morphological characteristics of rice lines that exhibit oxyfluorfen resistance, and to methods for producing a rice plant produced by crossing rice varieties that exhibit oxyfluorfen resistance with itself or with another rice line, and the creation of variants by mutagenesis, genetic modification or transformation of rice lines that exhibit oxyfluorfen resistance.

In one embodiment of the invention, there are provided novel rice plants containing mutant allele ROXY, which confers oxyfluorfen resistance. The present invention relates to rice lines containing mutant allele ROXY and having resistance to oxyfluorfen, including but not limited to rice lines designated “14G1”, “14G2”, “14G3”, “14G4”, “14G5”, “14G6”, “14G7”, “14G8”, “14G9”, “15G3”, and “15G4”. The present invention also relates to a rice seed, a rice plant, a rice line, and a rice hybrid containing mutant allele ROXY. The mutant allele ROXY present in these mutant lines has been determined to be a single recessive gene. The invention further provides plants, seeds, and other plant parts such as pollen and ovules containing the mutant allele ROXY. In addition, the present invention is directed to transferring mutant allele ROXY and oxyfluorfen resistance to other rice cultivars and species and is useful for producing rice cultivars and novel types with the oxyfluorfen resistance trait.

The invention also provides methods for introducing mutant allele ROXY of the present invention into other rice plants by crossing a rice plant which lacks the mutant allele with a rice plant that has mutant allele ROXY, selfing the resulting generations and then selecting the plants exhibiting, for example, one or more of the following: increased length of leaves, stem internodes and/or panicles and an increase in grain size of the rice plant, in addition to oxyfluorfen resistance. The invention further provides methods for introducing mutant allele ROXY of the present invention into other rice plants by crossing a rice plant which lacks the mutant allele with a rice plant that has mutant allele ROXY, selfing the resulting generations and then selecting the plants having mutant allele ROXY.

In another aspect, the invention provides a method for producing a hybrid rice seed containing mutant allele ROXY comprising crossing a first plant parent with a second plant parent and harvesting the resultant hybrid rice seed, wherein either one or both parents contain mutant allele ROXY. The hybrid rice seeds, plant and parts thereof produced by such method are also part of the invention.

In another aspect, the present invention provides for single or multiple gene converted plants containing mutant allele ROXY. The desired single or multiple transferred gene(s) may preferably be a dominant or recessive allele. Preferably, the single or multiple transferred gene(s) will confer such traits including but not limited to herbicide resistance, insect resistance, resistance to bacterial, fungal, or viral disease, male fertility, male sterility, enhanced nutritional quality, and industrial usage. The single or multiple gene(s) may be a naturally occurring rice gene or a transgene introduced through genetic engineering techniques.

The invention also relates to methods for producing a rice plant having mutant allele ROXY containing in its genetic material one or more transgenes and to the transgenic rice plant produced by those methods. The invention further relates to methods for genetically modifying a rice plant having mutant allele ROXY and to the modified rice plant produced by those methods. The genetic modification methods may include, but are not limited to mutation breeding, genome editing, backcross conversion, genetic transformation, single and multiple gene conversion, and/or direct gene transfer.

In another aspect, the present invention provides regenerable cells for use in tissue culture of a rice plant containing mutant allele ROXY. The tissue culture will preferably be capable of regenerating plants having the mutant allele ROXY and all of the physiological and morphological characteristics of the foregoing rice plant, and of regenerating plants having substantially the same genotype as the foregoing rice plant. Genetic variants of rice plants having resistance to oxyfluorfen and mutant allele ROXY naturally generated through using tissue culture or artificially induced utilizing mutagenic agents or genome editing techniques during tissue culture, are aspects of the present invention. Preferably, the regenerable cells in such tissue cultures will be embryos, protoplasts, meristematic cells, callus, pollen, leaves, anthers, pistils, roots, root tips, flowers, seeds, panicles or stems. Still further, the present invention provides rice plants regenerated from the tissue cultures of the invention.

The invention further provides methods for developing rice plants having mutant allele ROXY in a rice plant breeding program using plant breeding techniques including recurrent selection, backcrossing, pedigree breeding, restriction fragment length polymorphism enhanced selection, genetic marker enhanced selection, and transformation. Seeds, rice plants, and parts thereof, produced by such breeding methods are also part of the invention.

Another aspect of the invention relates to any rice seed or plant having mutant allele ROXY. A further aspect of the invention relates to any rice seed or plant having resistance to the herbicide oxyfluorfen.

In addition to the exemplary aspects and embodiments described above, further aspects and embodiments will become apparent by study of the following descriptions.

### BRIEF DESCRIPTION OF THE DRAWINGS

FIG. 1 shows the unexpected improved resistance to oxyfluorfen of lines 14G1 to 14G9 (1 to 9) containing mutant allele ROXY over M-206 without mutant allele ROXY, as reflected by the growth of the seedling (average seedling height). Unexpectedly, by the measurement of seedling height at 14 days, oxyfluorfen resistant rice lines 14G1 to 14G9 containing mutant allele ROXY were significantly taller than M-206 with the oxyfluorfen treatment at 1 pt./acre (280 g ai/ha) rate or higher.

FIG. 2 shows a photo of herbicide resistance seen in water-seeded plots treated at seeding with oxyfluorfen. Oxyfluorfen resistant rice lines of the present invention, 14G9 (left side) and 14G3 (right side), which contain mutant allele ROXY, grew through the oxyfluorfen treated water, whereas susceptible line M-206 (middle) had low seedling survival.

FIG. 3 shows the characteristic single gene bimodal frequency distribution for the F₂ population of the cross of 14G4×M-206 and the distribution of the parents for plant seedling height in millimeters (mm) after treatment with oxyfluorfen.

FIG. 4 shows the phenotypic classification of the results of FIG. 3 of the F₂ plants for short (oxyfluorfen susceptible) or tall (oxyfluorfen resistance).

FIG. 5 shows that the location of the mutant allele ROXY gene (orange mark) is flanked by markers RM3870 and RM3476 of Chromosome 5.

### SUMMARY OF THE SEQUENCE LISTING

The present application is being filed along with a Sequence Listing in electronic format. The Sequence Listing is entitled SequenceListing_ST25.txt, was created on 14 Jul. 2017 and is 1 kb in size. The information in the electronic format of the Sequence Listing is part of the present application and is incorporated herein by reference in its entirety.

SEQ ID NO:1 sets forth the sequence of the forward base primer for the flanking marker RM3870.

SEQ ID NO:2 sets forth the sequence of the reverse base primer for the flanking marker RM3870.

SEQ ID NO:3 sets forth the sequence of the forward base primer for the flanking marker RM3476.

SEQ ID NO:4 sets forth the sequence of the reverse base primer for the flanking marker RM3476.

### DETAILED DESCRIPTION OF THE INVENTION

In the description and tables which follow, a number of terms are used. In order to provide a clear and consistent understanding of the specification and claims, including the scope to be given such terms, the following definitions are provided:

Allele. An allele is any of one or more alternative forms of a gene which relate to one trait or characteristic. In a diploid cell or organism, the two alleles of a given gene occupy corresponding loci on a pair of homologous chromosomes.

Alter. The utilization of up-regulation, down-regulation, or gene silencing.

Backcrossing. Backcrossing is a process in which a breeder successively crosses hybrid progeny back to one of the parents, for example, a first generation hybrid F₁ with one of the parental genotypes of the F₁ hybrid.

Cell. Cell as used herein includes a plant cell, whether isolated, in tissue culture or incorporated in a plant or plant part.

Cotyledon. A cotyledon is a type of seed leaf. The cotyledon contains the food storage tissues of the seed.

Days to 50% heading. Average number of days from planting to the day when 50% of all panicles are exerted at least partially through the leaf sheath. A measure of maturity.

Embryo. The embryo is the small plant contained within a mature seed.

Essentially all the physiological and morphological characteristics. A plant having essentially all the physiological and morphological characteristics means a plant having the physiological and morphological characteristics of the cultivar, except for the characteristics derived from the converted gene.

g ai/ha. Grams of active ingredient applied per hectare, a standard unit of measure used in herbicide or insecticide research.

Genetically modified. Describes an organism that has received genetic material from another organism, or had its genetic material modified, resulting in a change in one or more of its phenotypic characteristics. Methods used to modify, introduce or delete the genetic material may include mutation breeding, genome editing, backcross conversion, genetic transformation, single and multiple gene conversion, and/or direct gene transfer.

Genome editing. A type of genetic engineering in which DNA is inserted, replaced, modified or removed from a genome using artificially engineered nucleases or other targeted changes using homologous recombination. Examples include but are not limited to use of zinc finger nucleases (ZFNs), TAL effector nucleases (TALENs) and CRISPR/Cas9. (Ma et. al., Molecular Plant, 9:961-974 (2016); Belhaj et. al., Current Opinion in Biotechnology, 32:76-84 (2015)).

Grain. Caryopsis of a cereal plant. In this case the rice grain, seed, often referred to as paddy rice. It includes the hull covering the brown rice kernel with intact bran layers and germ.

Gene Silencing. Gene silencing refers to the interruption or suppression of the expression of a gene at the level of transcription or translation.

Half diallel. Crossing scheme where a set of lines are crossed in all combinations, omitting reciprocal crosses.

Harvest Moisture. Harvest moisture refers to the percent of moisture of the grain when harvested.

Leaf. The rice leaf consist of a sheath and a blade (lamina). The leaf sheath is an elongated part of the leaf rolled into a cylinder that encloses the developing new leaves and stem at later growth stages. The basal portion of the leaf sheath is attached to a nodal plate. The leaf blade is long and lanceolate with a midrib and has parallel veins on each side.

Locus. A locus confers one or more traits such as, for example, male sterility, oxyfluorfen resistance trait, insect resistance, disease resistance, and improved yield. The trait may be, for example, conferred by a naturally occurring gene introduced into the genome of the variety by backcrossing, a natural or induced mutation, or a transgene introduced through genetic transformation techniques. A locus may comprise one or more alleles integrated at a single chromosomal location.

Lodging (also called Straw Strength). Lodging is a visual estimate of the percentage of the plot leaning or fallen completely to the ground before harvest.

M₁, M₂, M₃, etc. Used to indicate the generations after a mutational treatment, analogous to filial generation F₁, F₂, etc. that identifies generations after a hybridization of two individuals that are advanced through self-fertilization.

Multiple Gene Converted (Conversion). Multiple gene converted (conversion) includes plants developed by a plant breeding technique called backcrossing wherein essentially all of the desired morphological and physiological characteristics of a variety are recovered, while retaining two or more genes transferred into the variety via crossing and backcrossing. The term can also refer to the introduction of multiple genes through genetic engineering techniques known in the art.

Mutant allele ROXY. The mutant allele of the present invention which confers non-transgenic resistance to the herbicide oxyfluorfen and is found in the oxyfluorfen resistant rice lines of the present invention, including but not limited to, “14G1”, “14G2”, “14G3”, “14G4”, “14G5”, “14G6”, “14G7”, “14G8”, “14G9”, “15G3”, and “15G4”. Representative samples of seed containing mutant allele ROXY have been deposited under ATCC Accession Number PTA-123525. The oxyfluorfen resistance gene ROXY is inherited as a single, recessive gene and is located between markers RM3870 and RM3476 on rice Chromosome 5.

Oxyfluorfen. A selective pre- and post-emergent herbicide used to control certain annual broadleaf and grassy weeds in rice and other crops, having the molecular formula C₁₅H₁₁ClF₃NO₄. Oxyfluorfen is a contact herbicide and light is required for it to affect target plants. Some trade names of oxyfluorfen include Goal® 2XL, GoalTender®, Koltar® EC, Collide™, OxyStar® 2E, OxyStar® 4L and RH-2915. Oxyfluorfen is a member of the diphenyl ether group of herbicides. The mode of action of oxyfluorfen is to inhibit protoporphyrinogen oxidase (PPO or PPOase; also referred to as Protox); the PPO gene has been identified in the literature as a possible site that provides resistance for PPOase inhibiting herbicides.

Oxyfluorfen resistant rice lines. Oxyfluorfen resistant rice lines of the present invention include, but are not limited to, “14G1”, “14G2”, “14G3”, “14G4”, “14G5”, “14G6”, “14G7”, “14G8”, “14G9”, “15G3”, and “15G4”, which contain mutant allele ROXY. Representative samples of seed having oxyfluorfen resistance and containing mutant allele ROXY have been deposited under ATCC Accession Number PTA-123525.

Panicle. Panicle refers to the inflorescence of the rice plant.

Plant. As used herein, the term “plant” includes reference to an immature or mature whole plant, including a plant from which seed or grain or anthers have been removed. A seed or embryo that will produce the plant is also considered to be the plant.

Plant Height. Rice plant height is measured in centimeters from soil surface to the tip of the extended panicle at harvest.

Plant Parts. As used herein, the term “plant parts” (or a rice plant, or a part thereof) includes protoplasts, leaves, stems, roots, root tips, anthers, pistils, seed, grain, embryo, pollen, ovules, cotyledon, hypocotyl, panicles, flower, shoot, tissue, petiole, cells, meristematic cells and the like.

Plastids. Small, double-membraned organelles of plant cells that contain their own DNA and ribosomes. Some plastids, such as the chloroplasts in plant leaves, contain pigments used in photosynthesis.

Preflood. Prior to application of flood water to a rice paddy. ‘Preflood’ is a term used in reference to the timing of an activity, such as an herbicide or fertilizer application.

Quantitative Trait Loci (QTL). Quantitative trait loci (QTL) refer to genetic loci that control to some degree numerically representable traits that are usually continuously distributed.

Regeneration. Regeneration refers to the development of a plant from tissue culture.

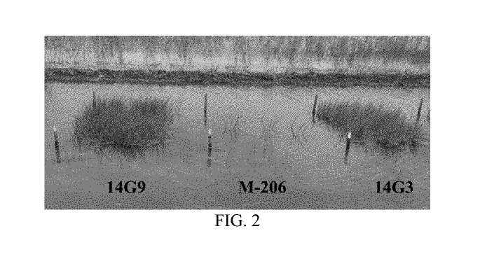

Resistance or resistant to oxyfluorfen. Refers to the ability of a seedling or plant not to be killed or damaged as the result of the application of the herbicide oxyfluorfen to the soil, water or plant surfaces. The rice lines of the present invention, which contain mutant allele ROXY, exhibit resistance to treatment with oxyfluorfen when compared to commercial rice varieties without ROXY grown in the same environment and receiving the same treatment with oxyfluorfen. For example, when compared to commercial rice varieties grown in the same environment and receiving the same treatment with oxyfluorfen, the oxyfluorfen resistant rice lines of the present invention containing mutant allele ROXY have significantly increased seedling vigor, better lodging resistance, and significantly increased grain production and yield. FIG. 2 shows a visual example of mutant rice lines resistant to oxyfluorfen (14G9 and 14G3 on left and right sides) compared to a non-resistant rice line without ROXY (M-206 in middle) grown in the same environment and receiving the same oxyfluorfen treatment.

Seedling emergence. The point at which the tip of the leaf of the growing rice seedling leaf emerges through the water in water seeded rice or the soil in direct seeded rice. This may be measured in days to seedling emergence as well as the number or percentage of seedlings that have emerged.

Seedling Vigor. Seedling vigor refers to the ability of the seedling to emerge rapidly through the soil or water after planting. It is frequently measured by visual observation field test and assigned a relative score.

Single Gene Converted (Conversion). Single gene converted (conversion) plant refers to plants which are developed by a plant breeding technique called backcrossing with selection wherein essentially all of the desired morphological and physiological characteristics of a variety are recovered in addition to the single gene transferred into the variety via the backcrossing technique or via genetic engineering.

Transgenic. Transgenic refers to plants that have been genetically engineered using recombinant DNA techniques to create plants with new characteristics. A transgenic organism is one that contains a gene or genes that have been artificially inserted instead of the organism acquiring them through reproduction.

Water seeding. Water seeding is the predominate planting method used in commercial rice production in California. Seeds are soaked in water (typically 24 hours) to initiate germination (24 to 48 hours) and seeded by aircraft in flooded field.

The present invention is directed towards rice plants that show enhanced resistance to the herbicide oxyfluorfen and the application of oxyfluorfen to improve weed control in water seeded rice fields and other possible uses. The present invention relates to a new and distinctive rice mutant allele designated ROXY, which confers non-transgenic resistance to the herbicide oxyfluorfen.

According to the invention, there are provided novel rice lines designated 14G1, 14G2, 14G3, 14G4, 14G5, 14G6, 14G7, 14G8, 14G9, 15G3, and 15G4 that are resistant to the herbicide oxyfluorfen and contain mutant allele ROXY, and results in rice seedlings with this trait having the ability to grow and emerge through the water in water-seeded rice where the soil or water has been treated pre-plant and/or pre-flood with oxyfluorfen and also when treated at the date of seeding while suppressing or controlling weeds. This invention thus relates to mutant allele ROXY, to rice seeds containing mutant allele ROXY, to rice plants containing mutant allele ROXY, and to methods for producing a rice plant by crossing a rice plant containing mutant allele ROXY with itself or another rice line.

Thus, any such methods using rice containing mutant allele ROXY are part of this invention: selfing, backcrosses, hybrid production, crosses to populations, and the like. All plants produced using rice containing mutant allele ROXY as a parent are within the scope of this invention.

The oxyfluorfen resistance conferred by mutant allele ROXY of the present invention is heritable and has been transferred to numerous different rice lines. Rice lines having mutant allele ROXY have shown uniformity and stability.

### Further Embodiments of the Invention

With the advent of molecular biological techniques that have allowed the isolation and characterization of genes that encode specific protein products, scientists in the field of plant biology developed a strong interest in engineering the genome of plants to contain and express foreign genes, or additional, or modified versions of native, or endogenous, genes (perhaps driven by different promoters) in order to alter the traits of a plant in a specific manner. Such foreign additional and/or modified genes are referred to herein collectively as “transgenes”. In some embodiments of the invention, a transgenic variant of rice plants containing mutant allele ROXY may contain at least one transgene but could contain at least 1, 2, 3, 4, 5, 6, 7, 8, 9, 10 and/or no more than 15, 14, 13, 12, 11, 10, 9, 8, 7, 6, 5, 4, 3, or 2. Over the last fifteen to twenty years, several methods for producing transgenic plants have been developed, and the present invention, in particular embodiments, also relates to transformed versions of the claimed cultivar.

Culture for expressing desired structural genes and cultured cells are known in the art. Also as known in the art, rice is transformable and regenerable such that whole plants containing and expressing desired genes under regulatory control may be obtained. General descriptions of plant expression vectors and reporter genes and transformation protocols can be found in Gruber, et al., “Vectors for Plant Transformation”, in Methods in Plant Molecular Biology & Biotechnology in Glich, et al., (Eds. pp. 89-119, CRC Press, 1993). Moreover GUS expression vectors and GUS gene cassettes are available from Clone Tech Laboratories, Inc., Palo Alto, Calif. while luciferase expression vectors and luciferase gene cassettes are available from Pro Mega Corp. (Madison, Wis.). General methods of culturing plant tissues are provided for example by Maki, et al., “Procedures for Introducing Foreign DNA into Plants” in Methods in Plant Molecular Biology & Biotechnology, Glich, et al., (Eds. pp. 67-88 CRC Press, 1993); and by Phillips, et al., “Cell-Tissue Culture and In-Vitro Manipulation” in Corn & Corn Improvement, 3rd Edition; Sprague, et al., (Eds. pp. 345-387 American Society of Agronomy Inc., 1988). Methods of introducing expression vectors into plant tissue include the direct infection or co-cultivation of plant cells with Agrobacterium tumefaciens, described for example by Horsch et al., Science, 227:1229 (1985). Descriptions of Agrobacterium vector systems and methods for Agrobacterium-mediated gene transfer are provided by Gruber, et al., supra.

Useful methods include but are not limited to expression vectors introduced into plant tissues using a direct gene transfer method such as microprojectile-mediated delivery, DNA injection, electroporation and the like. More preferably expression vectors are introduced into plant tissues using a microprojectile media delivery system with a biolistic device or using Agrobacterium-mediated transformation. Transformant plants obtained with the protoplasm of the invention are intended to be within the scope of this invention.

Plant transformation involves the construction of an expression vector which will function in plant cells. Such a vector comprises DNA comprising a gene under control of or operatively linked to a regulatory element (for example, a promoter). The expression vector may contain one or more such operably linked gene/regulatory element combinations. The vector(s) may be in the form of a plasmid, and can be used alone or in combination with other plasmids, to provide transformed rice plants, using transformation methods as described below to incorporate transgenes into the genetic material of the rice plant(s).

### Expression Vectors for Transformation: Marker Genes

Expression vectors include at least one genetic marker, operably linked to a regulatory element (a promoter, for example) that allows transformed cells containing the marker to be either recovered by negative selection, i.e., inhibiting growth of cells that do not contain the selectable marker gene, or by positive selection, i.e., screening for the product encoded by the genetic marker. Many commonly used selectable marker genes for plant transformation are well known in the transformation arts, and include, for example, genes that code for enzymes that metabolically detoxify a selective chemical agent which may be an antibiotic or an herbicide, or genes that encode an altered target which is insensitive to the inhibitor. A few positive selection methods are also known in the art.

One commonly used selectable marker gene for plant transformation is the neomycin phosphotransferase II (nptII) gene, isolated from transposon Tn5, which when placed under the control of plant regulatory signals confers resistance to kanamycin. Fraley et al., Proc. Natl. Acad. Sci. U.S.A., 80:4803 (1983). Another commonly used selectable marker gene is the hygromycin phosphotransferase gene which confers resistance to the antibiotic hygromycin. Vanden Elzen et al., Plant Mol. Biol., 5:299 (1985).

Additional selectable marker genes of bacterial origin that confer resistance to antibiotics include gentamycin acetyl transferase, streptomycin phosphotransferase, and aminoglycoside-3′-adenyl transferase, the bleomycin resistance determinant. Hayford et al., Plant Physiol. 86:1216 (1988); Jones et al., Mol. Gen. Genet., 210:86 (1987); Svab et al., Plant Mol. Biol. 14:197 (1990); Hille et al., Plant Mol. Biol. 7:171 (1986). Other selectable marker genes confer resistance to herbicides such as glyphosate, glufosinate or bromoxynil. Comai et al., Nature 317:741-744 (1985), Gordon-Kamm et al., Plant Cell 2:603-618 (1990) and Stalker et al., Science 242:419-423 (1988).

Selectable marker genes for plant transformation not of bacterial origin include, for example, mouse dihydrofolate reductase, plant 5-enolpyruvylshikimate-3-phosphate synthase and plant acetolactate synthase. Eichholtz et al., Somatic Cell Mol. Genet. 13:67 (1987), Shah et al., Science 233:478 (1986), Charest et al., Plant Cell Rep. 8:643 (1990).

Another class of marker genes for plant transformation requires screening of presumptively transformed plant cells rather than direct genetic selection of transformed cells for resistance to a toxic substance such as an antibiotic. These genes are particularly useful to quantify or visualize the spatial pattern of expression of a gene in specific tissues and are frequently referred to as reporter genes because they can be fused to a gene or gene regulatory sequence for the investigation of gene expression. Commonly used genes for screening presumptively transformed cells include β-glucuronidase (GUS, β-galactosidase, luciferase and chloramphenicol acetyltransferase. Jefferson, R. A., Plant Mol. Biol. Rep. 5:387 (1987), Teeri et al., EMBO J. 8:343 (1989), Koncz et al., Proc. Natl. Acad. Sci U.S.A. 84:131 (1987), DeBlock et al., EMBO J. 3:1681 (1984). Another approach to the identification of relatively rare transformation events has been use of a gene that encodes a dominant constitutive regulator of the Zea mays anthocyanin pigmentation pathway. Ludwig et al., Science 247:449 (1990).

In vivo methods for visualizing GUS activity that do not require destruction of plant tissue are available. Molecular Probes publication 2908, IMAGENE GREEN, p. 1-4 (1993) and Naleway et al., J. Cell Biol. 115:151a (1991). However, these in vivo methods for visualizing GUS activity have not proven useful for recovery of transformed cells because of low sensitivity, high fluorescent backgrounds and limitations associated with the use of luciferase genes as selectable markers.

More recently, a gene encoding Green Fluorescent Protein (GFP) has been utilized as a marker for gene expression in prokaryotic and eukaryotic cells. Chalfie et al., Science 263:802 (1994). GFP and mutants of GFP may be used as screenable markers.

### Expression Vectors for Transformation: Promoters

Genes included in expression vectors must be driven by nucleotide sequence comprising a regulatory element, for example, a promoter. Several types of promoters are now well known in the transformation arts, as are other regulatory elements that can be used alone or in combination with promoters.

As used herein, “promoter” includes reference to a region of DNA upstream from the start of transcription and involved in recognition and binding of RNA polymerase and other proteins to initiate transcription. A “plant promoter” is a promoter capable of initiating transcription in plant cells. Examples of promoters under developmental control include promoters that preferentially initiate transcription in certain tissues, such as leaves, roots, seeds, fibers, xylem vessels, tracheids, or sclerenchyma. Such promoters are referred to as “tissue-preferred”. Promoters which initiate transcription only in certain tissue are referred to as “tissue-specific”. A “cell type” specific promoter primarily drives expression in certain cell types in one or more organs, for example, vascular cells in roots or leaves. An “inducible” promoter is a promoter which is under environmental control. Examples of environmental conditions that may affect transcription by inducible promoters include anaerobic conditions or the presence of light. Tissue-specific, tissue-preferred, cell type specific, and inducible promoters constitute the class of “non-constitutive” promoters. A “constitutive” promoter is a promoter which is active under most environmental conditions.

A. Inducible Promoters—An inducible promoter is operably linked to a gene for expression in rice. Optionally, the inducible promoter is operably linked to a nucleotide sequence encoding a signal sequence which is operably linked to a gene for expression in rice. With an inducible promoter the rate of transcription increases in response to an inducing agent.

Any inducible promoter can be used in the instant invention. See Ward et al., Plant Mol. Biol. 22:361-366 (1993). Exemplary inducible promoters include, but are not limited to, that from the ACEI system which responds to copper (Meft et al., PNAS 90:4567-4571 (1993)); In2 gene from maize which responds to benzenesulfonamide herbicide safeners (Hershey et al., Mol. Gen Genetics 227:229-237 (1991) and Gatz et al., Mol. Gen. Genetics 243:32-38 (1994)) or Tet repressor from Tn10 (Gatz et al., Mol. Gen. Genetics 227:229-237 (1991)). A particularly preferred inducible promoter is a promoter that responds to an inducing agent to which plants do not normally respond. An exemplary inducible promoter is the inducible promoter from a steroid hormone gene, the transcriptional activity of which is induced by a glucocorticosteroid hormone (Schena et al., Proc. Natl. Acad. Sci. U.S.A. 88:0421 (1991)).

B. Constitutive Promoters—A constitutive promoter is operably linked to a gene for expression in rice or the constitutive promoter is operably linked to a nucleotide sequence encoding a signal sequence which is operably linked to a gene for expression in rice.

Many different constitutive promoters can be utilized in the instant invention. Exemplary constitutive promoters include, but are not limited to, the promoters from plant viruses such as the 35S promoter from CaMV (Odell et al., Nature 313:810-812 (1985)) and the promoters from such genes as rice actin (McElroy et al., Plant Cell 2:163-171 (1990)); ubiquitin (Christensen et al., Plant Mol. Biol. 12:619-632 (1989) and Christensen et al., Plant Mol. Biol. 18:675-689 (1992)); pEMU (Last et al., Theor. Appl. Genet. 81:581-588 (1991)); MAS (Velten et al., EMBO J. 3:2723-2730 (1984)) and maize H3 histone (Lepetit et al., Mol. Gen. Genetics 231:276-285 (1992) and Atanassova et al., Plant Journal 2 (3): 291-300 (1992)).

The ALS promoter, Xba1/Nco1 fragment 5′ to the Brassica napus ALS3 structural gene (or a nucleotide sequence similarity to said Xba1/Nco1 fragment), represents a particularly useful constitutive promoter. See PCT application WO96/30530.

C. Tissue-specific or Tissue-preferred Promoters—A tissue-specific promoter is operably linked to a gene for expression in rice. Optionally, the tissue-specific promoter is operably linked to a nucleotide sequence encoding a signal sequence which is operably linked to a gene for expression in rice. Plants transformed with a gene of interest operably linked to a tissue-specific promoter produce the protein product of the transgene exclusively, or preferentially, in a specific tissue.

Any tissue-specific or tissue-preferred promoter can be utilized in the instant invention. Exemplary tissue-specific or tissue-preferred promoters include, but are not limited to, a root-preferred promoter, such as that from the phaseolin gene (Murai et al., Science 23:476-482 (1983) and Sengupta-Gopalan et al., Proc. Natl. Acad. Sci. U.S.A. 82:3320-3324 (1985)); a leaf-specific and light-induced promoter such as that from cab or rubisco (Simpson et al., EMBO 4(11):2723-2729 (1985) and Timko et al., Nature 318:579-582 (1985)); an anther-specific promoter such as that from LAT52 (Twell et al., Mol. Gen. Genetics 217:240-245 (1989)); a pollen-specific promoter such as that from Zm13 (Guerrero et al., Mol. Gen. Genetics 244:161-168 (1993)) or a microspore-preferred promoter such as that from apg (Twell et al., Sex. Plant Reprod. 6:217-224 (1993)).

### Signal Sequences for Targeting Proteins to Subcellular Compartments

Transport of protein produced by transgenes to a subcellular compartment such as the chloroplast, vacuole, peroxisome, glyoxysome, cell wall or mitochondrion or for secretion into the apoplast, is accomplished by means of operably linking the nucleotide sequence encoding a signal sequence to the 5′ and/or 3′ region of a gene encoding the protein of interest. Targeting sequences at the 5′ and/or 3′ end of the structural gene may determine, during protein synthesis and processing, where the encoded protein is ultimately compartmentalized.

The presence of a signal sequence directs a polypeptide to either an intracellular organelle or subcellular compartment or for secretion to the apoplast. Many signal sequences are known in the art. See, for example Becker et al., Plant Mol. Biol. 20:49 (1992); Close, P. S., Master's Thesis, Iowa State University (1993); Knox, C., et al., Plant Mol. Biol. 9:3-17 (1987); Lerner et al., Plant Physiol. 91:124-129 (1989); Fontes et al., Plant Cell 3:483-496 (1991); Matsuoka et al., Proc. Natl. Acad. Sci. 88:834 (1991); Gould et al., J. Cell. Biol. 108:1657 (1989); Creissen et al., Plant 2:129 (1991); Kalderon, et al., Cell 39:499-509 (1984); Steifel, et al., Plant Cell 2:785-793 (1990).

### Foreign Protein Genes and Agronomic Genes

With transgenic plants according to the present invention, a foreign protein can be produced in commercial quantities. Thus, techniques for the selection and propagation of transformed plants, which are well understood in the art, yield a plurality of transgenic plants which are harvested in a conventional manner, and a foreign protein then can be extracted from a tissue of interest or from total biomass. Protein extraction from plant biomass can be accomplished by known methods which are discussed, for example, by Heney and Orr, Anal. Biochem. 114:92-6 (1981).

According to an embodiment, the transgenic plant provided for commercial production of foreign protein is rice. In another embodiment, the biomass of interest is seed. For the relatively small number of transgenic plants that show higher levels of expression, a genetic map can be generated, primarily via conventional RFLP, PCR and SSR analysis, which identifies the approximate chromosomal location of the integrated DNA molecule. For exemplary methodologies in this regard, see Glick and Thompson, Methods in Plant Molecular Biology and Biotechnology CRC Press, Boca Raton 269:284 (1993).

Map information concerning chromosomal location is useful for proprietary protection of a subject transgenic plant. If unauthorized propagation is undertaken and crosses made with other germplasm, the map of the integration region can be compared to similar maps for suspect plants, to determine if the latter have a common parentage with the subject plant. Map comparisons would involve hybridizations, RFLP, PCR, SSR and sequencing, all of which are conventional techniques.

Through the transformation of rice, the expression of genes can be altered to enhance disease resistance, insect resistance, herbicide resistance, agronomic quality and other traits. Transformation can also be used to insert DNA sequences which control or help control male-sterility. DNA sequences native to rice as well as non-native DNA sequences can be transformed into rice and used to alter levels of native or non-native proteins. Various promoters, targeting sequences, enhancing sequences, and other DNA sequences can be inserted into the genome for the purpose of altering the expression of proteins. Reduction of the activity of specific genes (also known as gene silencing, or gene suppression) is desirable for several aspects of genetic engineering in plants.

Many techniques for gene silencing are well known to one of skill in the art, including but not limited to knock-outs (such as by insertion of a transposable element such as mu (Vicki Chandler, The Maize Handbook ch. 118 (Springer-Verlag 1994) or other genetic elements such as a FRT, Lox or other site specific integration site, antisense technology (see, e.g., Sheehy et al. (1988) PNAS USA 85:8805-8809; and U.S. Pat. Nos. 5,107,065; 5,453,566; and 5,759,829); co-suppression (e.g., Taylor (1997) Plant Cell 9:1245; Jorgensen (1990) Trends Biotech. 8(12):340-344; Flavell (1994) PNAS USA 91:3490-3496; Finnegan et al. (1994) Bio/Technology 12: 883-888; and Neuhuber et al. (1994) Mol. Gen. Genet. 244:230-241); RNA interference (Napoli et al. (1990) Plant Cell 2:279-289; U.S. Pat. No. 5,034,323; Sharp (1999) Genes Dev. 13:139-141; Zamore et al. (2000) Cell 101:25-33; and Montgomery et al. (1998) PNAS USA 95:15502-15507), virus-induced gene silencing (Burton, et al. (2000) Plant Cell 12:691-705; and Baulcombe (1999) Curr. Op. Plant Bio. 2:109-113); target-RNA-specific ribozymes (Haseloff et al. (1988) Nature 334: 585-591); hairpin structures (Smith et al. (2000) Nature 407:319-320; WO 99/53050; and WO 98/53083); MicroRNA (Aukerman& Sakai (2003) Plant Cell 15:2730-2741); ribozymes (Steinecke et al. (1992) EMBO J. 11:1525; and Perriman et al. (1993) Antisense Res. Dev. 3:253); oligonucleotide mediated targeted modification (e.g., WO 03/076574 and WO 99/25853); Zn-finger targeted molecules (e.g., WO 01/52620; WO 03/048345; and WO 00/42219); and other methods or combinations of the above methods known to those of skill in the art.

Likewise, by means of the present invention, agronomic genes can be expressed in transformed plants. More particularly, plants can be genetically engineered to express various phenotypes of agronomic interest. Exemplary genes implicated in this regard include, but are not limited to, those categorized below:

### 1. Genes That Confer Resistance to Pests or Disease and That Encode:

A. Plant disease resistance genes. Plant defenses are often activated by specific interaction between the product of a disease resistance gene (R) in the plant and the product of a corresponding avirulence (Avr) gene in the pathogen. A plant cultivar can be transformed with cloned resistance gene to engineer plants that are resistant to specific pathogen strains. See, for example Jones et al., Science 266:789 (1994) (cloning of the tomato Cf-9 gene for resistance to Cladosporium fulvum); Martin et al., Science 262:1432 (1993) (tomato Pto gene for resistance to Pseudomonas syringae pv. tomato encodes a protein kinase); Mindrinos et al., Cell 78:1089 (1994) (Arabidopsis RSP2 gene for resistance to Pseudomonas syringae).

B. A Bacillus thuringiensis protein, a derivative thereof or a synthetic polypeptide modeled thereon. See, for example, Geiser et al., Gene 48:109 (1986), who disclose the cloning and nucleotide sequence of a Bt δ-endotoxin gene. Moreover, DNA molecules encoding 6-endotoxin genes can be purchased from American Type Culture Collection, Manassas, Va., for example, under ATCC Accession Nos. 40098, 67136, 31995 and 31998.

C. A lectin. See, for example, the disclosure by Van Damme et al., Plant Molec. Biol. 24:25 (1994), who disclose the nucleotide sequences of several Clivia miniata mannose-binding lectin genes.

D. A vitamin-binding protein such as avidin. See PCT application US93/06487. The application teaches the use of avidin and avidin homologues as larvicides against insect pests.

E. An enzyme inhibitor, for example, a protease or proteinase inhibitor or an amylase inhibitor. See, for example, Abe et al., J. Biol. Chem. 262:16793 (1987) (nucleotide sequence of rice cysteine proteinase inhibitor), Huub et al., Plant Molec. Biol. 21:985 (1993) (nucleotide sequence of cDNA encoding tobacco proteinase inhibitor I), Sumitani et al., Biosci. Biotech. Biochem. 57:1243 (1993) (nucleotide sequence of Streptomyces nitrosporeus α-amylase inhibitor).

F. An insect-specific hormone or pheromone such as an ecdysteroid and juvenile hormone, a variant thereof, a mimetic based thereon, or an antagonist or agonist thereof. See, for example, the disclosure by Hammock et al., Nature 344:458 (1990), of baculovirus expression of cloned juvenile hormone esterase, an inactivator of juvenile hormone.

G. An insect-specific peptide or neuropeptide which, upon expression, disrupts the physiology of the affected pest. For example, see the disclosures of Regan, J. Biol. Chem. 269:9 (1994) (expression cloning yields DNA coding for insect diuretic hormone receptor), and Pratt et al., Biochem. Biophys. Res. Comm. 163:1243 (1989) (an allostatin is identified in Diploptera puntata). See also U.S. Pat. No. 5,266,317 to Tomalski et al., who disclose genes encoding insect-specific, paralytic neurotoxins.

H. An insect-specific venom produced in nature by a snake, a wasp, etc. For example, see Pang et al., Gene 116:165 (1992), for disclosure of heterologous expression in plants of a gene coding for a scorpion insectotoxic peptide.

I. An enzyme responsible for a hyper-accumulation of a monoterpene, a sesquiterpene, a steroid, a hydroxamic acid, a phenylpropanoid derivative or another non-protein molecule with insecticidal activity.

J. An enzyme involved in the modification, including the post-translational modification, of a biologically active molecule; for example, a glycolytic enzyme, a proteolytic enzyme, a lipolytic enzyme, a nuclease, a cyclase, a transaminase, an esterase, a hydrolase, a phosphatase, a kinase, a phosphorylase, a polymerase, an elastase, a chitinase and a glucanase, whether natural or synthetic. See PCT application WO 93/02197 in the name of Scott et al., which discloses the nucleotide sequence of a callase gene. DNA molecules which contain chitinase-encoding sequences can be obtained, for example, from the ATCC under Accession Nos. 39637 and 67152. See also Kramer et al., Insect Biochem. Molec. Biol. 23:691 (1993), who teach the nucleotide sequence of a cDNA encoding tobacco hornworm chitinase, and Kawalleck et al., Plant Molec. Biol. 21:673 (1993), who provide the nucleotide sequence of the parsley ubi4-2 polyubiquitin gene.

K. A molecule that stimulates signal transduction. For example, see the disclosure by Botella et al., Plant Molec. Biol. 24:757 (1994), of nucleotide sequences for mung bean calmodulin cDNA clones, and Griess et al., Plant Physiol. 104:1467 (1994), who provide the nucleotide sequence of a maize calmodulin cDNA clone.

L. A hydrophobic moment peptide. See PCT application WO95/16776 (disclosure of peptide derivatives of Tachyplesin which inhibit fungal plant pathogens) and PCT application WO95/18855 (teaches synthetic antimicrobial peptides that confer disease resistance), the respective contents of which are hereby incorporated by reference.

M. A membrane permease, a channel former or a channel blocker. For example, see the disclosure of Jaynes et al., Plant Sci 89:43 (1993), of heterologous expression of a cecropin-β, lytic peptide analog to render transgenic tobacco plants resistant to Pseudomonas solanacearum.

N. A viral-invasive protein or a complex toxin derived therefrom. For example, the accumulation of viral coat proteins in transformed plant cells imparts resistance to viral infection and/or disease development effected by the virus from which the coat protein gene is derived, as well as by related viruses. See Beachy et al., Ann. Rev. Phytopathol. 28:451 (1990). Coat protein-mediated resistance has been conferred upon transformed plants against alfalfa mosaic virus, cucumber mosaic virus, tobacco streak virus, potato virus X, potato virus Y, tobacco etch virus, tobacco rattle virus and tobacco mosaic virus. Id.

O. An insect-specific antibody or an immunotoxin derived therefrom. Thus, an antibody targeted to a critical metabolic function in the insect gut would inactivate an affected enzyme, killing the insect. Cf. Taylor et al., Abstract #497, Seventh Int'l Symposium on Molecular Plant-Microbe Interactions (Edinburgh, Scotland) (1994) (enzymatic inactivation in transgenic tobacco via production of single-chain antibody fragments).

P. A virus-specific antibody. See, for example, Tavladoraki et al., Nature 366:469 (1993), who show that transgenic plants expressing recombinant antibody genes are protected from virus attack.

Q. A developmental-arrestive protein produced in nature by a pathogen or a parasite. Thus, fungal endo-α-1, 4-D-polygalacturonases facilitate fungal colonization and plant nutrient release by solubilizing plant cell wall homo-α-1,4-D-galacturonase. See Lamb et al., Bio/Technology 10:1436 (1992). The cloning and characterization of a gene which encodes a bean endopolygalacturonase-inhibiting protein is described by Toubart et al., Plant J. 2:367 (1992).

R. A developmental-arrestive protein produced in nature by a plant. For example, Logemann et al., Bio/Technology 10:305 (1992), have shown that transgenic plants expressing the barley ribosome-inactivating gene have an increased resistance to fungal disease.

2. Genes That Confer Resistance to an Herbicide, for example:

A. An herbicide that inhibits the growing point or meristem, such as an imidazolinone or a sulfonylurea. Exemplary genes in this category code for mutant ALS and AHAS enzyme as described, for example, by Lee et al., EMBO J. 7:1241 (1988), and Miki et al., Theor. Appl. Genet. 80:449 (1990), respectively.

B. Glyphosate (resistance conferred by mutant 5-enolpyruvlshikimate-3-phosphate synthase (EPSP) and aroA genes, respectively) and other phosphono compounds such as glufosinate (phosphinothricin acetyl transferase (PAT) and Streptomyces hygroscopicus PAT, bar, genes), and pyridinoxy or phenoxy propionic acids and cyclohexones, as well as herbicides that inhibit the enzyme acetyl-CoA carboxylase (ACCase inhibitor-encoding genes). See, for example, U.S. Pat. No. 4,940,835 to Shah, et al., which discloses the nucleotide sequence of a form of EPSP which can confer glyphosate resistance. A DNA molecule encoding a mutant aroA gene can be obtained under ATCC accession number 39256, and the nucleotide sequence of the mutant gene is disclosed in U.S. Pat. No. 4,769,061 to Comai. European patent application No. 0 333 033 to Kumada et al., and U.S. Pat. No. 4,975,374 to Goodman et al., disclose nucleotide sequences of glutamine synthetase genes which confer resistance to herbicides such as L-phosphinothricin. The nucleotide sequence of a PAT gene is provided in European application No. 0 242 246 to Leemans et al. DeGreef et al., Bio/Technology 7:61 (1989), describe the production of transgenic plants that express chimeric bar genes coding for PAT activity. Exemplary of genes conferring resistance to phenoxy propionic acids and cyclohexones, such as sethoxydim and haloxyfop are the Acc1-S1, Acc1-S2 and Acc1-S3 genes described by Marshall et al., Theor. Appl. Genet. 83:435 (1992).

C. An herbicide that inhibits photosynthesis, such as a triazine (psbA and gs+ genes) or a benzonitrile (nitrilase gene). Przibilla et al., Plant Cell 3:169 (1991), describe the transformation of Chlamydomonas with plasmids encoding mutant psbA genes. Nucleotide sequences for nitrilase genes are disclosed in U.S. Pat. No. 4,810,648 to Stalker, and DNA molecules containing these genes are available under ATCC Accession Nos. 53435, 67441, and 67442. Cloning and expression of DNA coding for a glutathione S-transferase is described by Hayes et al., Biochem. J. 285:173 (1992).

### 3. Genes That Confer or Contribute to a Value-Added Trait, Such as:

A. Modified fatty acid metabolism, for example, by transforming a plant with an antisense gene of stearyl-ACP desaturase to increase stearic acid content of the plant. See Knultzon et al., Proc. Natl. Acad. Sci. U.S.A. 89:2624 (1992).

B. Decreased phytate content, 1) Introduction of a phytase-encoding gene would enhance breakdown of phytate, adding more free phosphate to the transformed plant. For example, see Van Hartingsveldt et al., Gene 127:87 (1993), for a disclosure of the nucleotide sequence of an Aspergillus niger phytase gene; 2) A gene could be introduced that reduced phytate content. In maize, this, for example, could be accomplished, by cloning and then reintroducing DNA associated with the single allele which is responsible for maize mutants characterized by low levels of phytic acid. See Raboy et al., Maydica 35:383 (1990).

C. Modified carbohydrate composition effected, for example, by transforming plants with a gene coding for an enzyme that alters the branching pattern of starch. See Shiroza et al., J. Bacteol. 170:810 (1988) (nucleotide sequence of Streptococcus mutants fructosyltransferase gene), Steinmetz et al., Mol. Gen. Genet. 20:220 (1985) (nucleotide sequence of Bacillus subtilis levansucrase gene), Pen et al., Bio/Technology 10:292 (1992) (production of transgenic plants that express Bacillus lichenifonnis α-amylase), Elliot et al., Plant Molec. Biol. 21:515 (1993) (nucleotide sequences of tomato invertase genes), SΠgaard et al., J. Biol. Chem. 268:22480 (1993) (site-directed mutagenesis of barley α-amylase gene), and Fisher et al., Plant Physiol. 102:1045 (1993) (maize endosperm starch branching enzyme II).

### Methods for Transformation

Numerous methods for plant transformation have been developed, including biological and physical, plant transformation protocols. See, for example, Miki et al., “Procedures for Introducing Foreign DNA into Plants” in Methods in Plant Molecular Biology and Biotechnology, Glick B. R. and Thompson, J. E. Eds. (CRC Press, Inc., Boca Raton, 1993) pages 67-88. In addition, expression vectors and in vitro culture methods for plant cell or tissue transformation and regeneration of plants are available. See, for example, Gruber et al., “Vectors for Plant Transformation” in Methods in Plant Molecular Biology and Biotechnology, Glick B. R. and Thompson, J. E. Eds. (CRC Press, Inc., Boca Raton, 1993) pages 89-119.

A. Agrobacterium-mediated Transformation—One method for introducing an expression vector into plants is based on the natural transformation system of Agrobacterium. See, for example, Horsch et al., Science 227:1229 (1985). A. tumefaciens and A. rhizogenes are plant pathogenic soil bacteria which genetically transform plant cells. The Ti and Ri plasmids of A. tumefaciens and A. rhizogenes, respectively, carry genes responsible for genetic transformation of the plant. See, for example, Kado, C. I., Crit. Rev. Plant Sci. 10:1 (1991). Descriptions of Agrobacterium vector systems and methods for Agrobacterium-mediated gene transfer are provided by Gruber et al., supra, Miki et al., supra, and Moloney et al., Plant Cell Reports 8:238 (1989). See also, U.S. Pat. No. 5,591,616 issued Jan. 7, 1997.

B. Direct Gene Transfer—Despite the fact the host range for Agrobacterium-mediated transformation is broad, some major cereal crop species and gymnosperms have generally been recalcitrant to this mode of gene transfer, even though some success has recently been achieved in rice and corn. Hiei et al., The Plant Journal 6:271-282 (1994) and U.S. Pat. No. 5,591,616 issued Jan. 7, 1997. Several methods of plant transformation, collectively referred to as direct gene transfer, have been developed as an alternative to Agrobacterium-mediated transformation.

A generally applicable method of plant transformation is microprojectile-mediated transformation wherein DNA is carried on the surface of microprojectiles measuring 1 to 4 μm. The expression vector is introduced into plant tissues with a biolistic device that accelerates the microprojectiles to speeds of 300 to 600 m/s which is sufficient to penetrate plant cell walls and membranes. Sanford et al., Part. Sci. Technol. 5:27 (1987), Sanford, J. C., Trends Biotech. 6:299 (1988), Klein et al., Bio/Technology 6:559-563 (1988), Sanford, J. C., Physiol Plant 7:206 (1990), Klein et al., Biotechnology 10:268 (1992). In corn, several target tissues can be bombarded with DNA-coated microprojectiles in order to produce transgenic plants, including, for example, callus (Type I or Type II), immature embryos, and meristematic tissue.

Another method for physical delivery of DNA to plants is sonication of target cells. Zhang et al., Bio/Technology 9:996 (1991). Additionally, liposome and spheroplast fusion have been used to introduce expression vectors into plants. Deshayes et al., EMBO 1, 4:2731 (1985), Christou et al., Proc Natl. Acad. Sci. U.S.A. 84:3962 (1987). Direct uptake of DNA into protoplasts using CaCl₂ precipitation, polyvinyl alcohol or poly-L-ornithine has also been reported. Hain et al., Mol. Gen. Genet. 199:161 (1985) and Draper et al., Plant Cell Physiol. 23:451 (1982). Electroporation of protoplasts and whole cells and tissues have also been described. Donn et al., In Abstracts of VIIth International Congress on Plant Cell and Tissue Culture IAPTC, A2-38, p 53 (1990); D'Halluin et al., Plant Cell 4:1495-1505 (1992) and Spencer et al., Plant Mol. Biol. 24:51-61 (1994).

Following transformation of rice target tissues, expression of the above-described selectable marker genes allows for preferential selection of transformed cells, tissues and/or plants, using regeneration and selection methods now well known in the art.

The foregoing methods for transformation would typically be used for producing a transgenic cultivar. The transgenic cultivar could then be crossed, with another (non-transformed or transformed) cultivar, in order to produce a new transgenic cultivar. Alternatively, a genetic trait which has been engineered into a particular rice cultivar using the foregoing transformation techniques could be moved into another cultivar using traditional backcrossing techniques that are well known in the plant breeding arts. For example, a backcrossing approach could be used to move an engineered trait from a public, non-elite cultivar into an elite cultivar, or from a cultivar containing a foreign gene in its genome into a cultivar which does not contain that gene. As used herein, “crossing” can refer to a simple X by Y cross, or the process of backcrossing, depending on the context.

### Single or Multiple Gene Conversion

When the term rice plant is used in the context of the present invention, this also includes any single or multiple gene conversions of that plant. The terms single or multiple gene converted plant as used herein refers to those rice plants which are developed by a plant breeding technique called backcrossing wherein essentially all of the desired morphological and physiological characteristics of a cultivar are recovered in addition to the single or multiple gene(s) transferred into the cultivar via the backcrossing technique. Backcrossing methods can be used with the present invention to improve or introduce a characteristic into the cultivar. The term backcrossing as used herein refers to the repeated crossing of a hybrid progeny back to one of the parental rice plants, the recurrent parent, for that cultivar, i.e., backcrossing 1, 2, 3, 4, 5, 6, 7, 8, 9 or more times to the recurrent parent. The parental rice plant which contributes the gene for the desired characteristic is termed the nonrecurrent or donor parent. This terminology refers to the fact that the nonrecurrent parent is used one time in the backcross protocol and therefore does not recur. The parental rice plant to which the gene or genes from the nonrecurrent parent are transferred is known as the recurrent parent as it is used for several rounds in the backcrossing protocol (Jennings, P. R. et al. Rice Improvement (1979); Mackill D. On your mark, get, select. Rice Today, July-September pp 28-29 (2004); Fehr, W. R. et al. Principles of Cultivar Development—Theory and Technique pp. 261-286 (1987) and Pohelman and Sleper (1994)).

In a typical backcross protocol, the original cultivar of interest (recurrent parent) is crossed to a second cultivar (nonrecurrent parent) that carries the single or multiple gene(s) of interest to be transferred. The resulting progeny from this cross are then crossed again to the recurrent parent and the process is repeated until a rice plant is obtained wherein essentially all of the desired morphological and physiological characteristics of the recurrent parent are recovered in the converted plant, in addition to the single or multiple transferred gene(s) from the nonrecurrent parent as determined at the 5% significance level when grown in the same environmental conditions.

The selection of a suitable recurrent parent is an important step for a successful backcrossing procedure. The goal of a backcross protocol is to alter or substitute a single or multiple trait or characteristic in the original cultivar. To accomplish this, a single or multiple gene(s) of the recurrent cultivar is modified or substituted with the desired gene from the nonrecurrent parent, while retaining essentially all of the rest of the desired genetic, and therefore the desired physiological and morphological, constitution of the original cultivar. The choice of the particular nonrecurrent parent will depend on the purpose of the backcross; one of the major purposes is to add some commercially desirable, agronomically important trait to the plant. The exact backcrossing protocol will depend on the characteristic or trait being altered to determine an appropriate testing protocol. Although backcrossing methods are simplified when the characteristic being transferred is a dominant allele, a recessive allele may also be transferred. In this instance it may be necessary to introduce a test of the progeny to determine if the desired characteristic has been successfully transferred.

Many single or multiple gene traits have been identified that are not regularly selected for in the development of a new cultivar but that can be improved by backcrossing techniques. Single or multiple gene traits may or may not be transgenic, examples of these traits include but are not limited to, male sterility, waxy starch, herbicide resistance, resistance for bacterial, fungal, or viral disease, insect resistance, male fertility, enhanced nutritional quality, industrial usage, yield stability and yield enhancement. These genes are generally inherited through the nucleus. Some known exceptions to this are the genes for male sterility, some of which are inherited cytoplasmically, but still act as single gene traits. Several of these single gene traits are described in U.S. Pat. Nos. 5,777,196; 5,948,957 and 5,969,212, the disclosures of which are specifically hereby incorporated by reference.

### Tissue Culture

Further reproduction of rice plants containing mutant allele ROXY, the oxyfluorfen resistance trait, can occur by tissue culture and regeneration. Tissue culture of various tissues of rice and regeneration of plants therefrom is well known and widely published. For example, reference may be had to Komatsuda, T. et al., Crop Sci. 31:333-337 (1991); Stephens, P. A., et al., Theor. Appl. Genet. (1991) 82:633-635; Komatsuda, T. et al., Plant Cell, Tissue and Organ Culture, 28:103-113 (1992); Dhir, S. et al., Plant Cell Reports (1992) 11:285-289; Pandey, P. A., et al., Japan J. Breed. 42:1-5 (1992); and Shetty, K., et al., Plant Science 81:245-251 (1992); as well as U.S. Pat. No. 5,024,944 issued Jun. 18, 1991 to Collins et al., and U.S. Pat. No. 5,008,200 issued Apr. 16, 1991 to Ranch et al. Thus, another aspect of this invention is to provide cells which upon growth and differentiation produce rice plants having the physiological and morphological characteristics of rice plants containing mutant allele ROXY.

As used herein, the term “tissue culture” indicates a composition comprising isolated cells of the same or a different type or a collection of such cells organized into parts of a plant. Exemplary types of tissue cultures are protoplasts, calli, plant clumps, and plant cells that can generate tissue culture that are intact in plants or parts of plants, such as embryos, pollen, flowers, seeds, pods, leaves, stems, roots, root tips, anthers, and the like. Means for preparing and maintaining plant tissue culture are well known in the art. By way of example, a tissue culture comprising organs has been used to produce regenerated plants. U.S. Pat. Nos. 5,959,185; 5,973,234 and 5,977,445 describe certain techniques, the disclosures of which are incorporated herein by reference.

As used herein, the term “plant” includes plant cells, plant protoplasts, plant cells of tissue culture from which rice plants can be regenerated, plant calli, plant clumps, and plant cells that are intact in plants or parts of plants, such as pollen, flowers, embryos, ovules, seeds, pods, leaves, stems, pistils, anthers and the like.

The present invention contemplates a rice plant regenerated from a tissue culture of a variety or hybrid plant having mutant allele ROXY of the present invention. As is well known in the art, tissue culture of rice can be used for the in vitro regeneration of a rice plant. Tissue culture of various tissues of rice and regeneration of plants therefrom is well known and widely published. For example, reference may be had to Chu, Q. R., et al., (1999) “Use of bridging parents with high anther culturability to improve plant regeneration and breeding value in rice”, Rice Biotechnology Quarterly 38:25-26; Chu, Q. R., et al., (1998), “A novel plant regeneration medium for rice anther culture of Southern U.S. crosses”, Rice Biotechnology Quarterly 35:15-16; Chu, Q. R., et al., (1997), “A novel basal medium for embryogenic callus induction of Southern US crosses”, Rice Biotechnology Quarterly 32:19-20; and Oono, K., “Broadening the Genetic Variability By Tissue Culture Methods”, Jap. J. Breed. 33 (Suppl. 2), 306-307, illus. 1983. Thus, another aspect of this invention is to provide cells which upon growth and differentiation produce rice plants having the physiological and morphological characteristics of rice plants containing mutant allele ROXY.

Duncan, et al., Planta 165:322-332 (1985) reflects that 97% of the plants cultured that produced callus were capable of plant regeneration. Subsequent experiments with both cultivars and hybrids produced 91% regenerable callus that produced plants. In a further study in 1988, Songstad, et al., Plant Cell Reports 7:262-265 (1988), reports several media additions that enhance regenerability of callus of two cultivars. Other published reports also indicated that “non-traditional” tissues are capable of producing somatic embryogenesis and plant regeneration. K. P. Rao et al., Maize Genetics Cooperation Newsletter, 60:64-65 (1986), refers to somatic embryogenesis from glume callus cultures and B. V. Conger, et al., Plant Cell Reports, 6:345-347 (1987) indicates somatic embryogenesis from the tissue cultures of corn leaf segments. Thus, it is clear from the literature that the state of the art is such that these methods of obtaining plants are routinely used and have a very high rate of success.

### Additional Breeding Methods

Although specific breeding objectives vary somewhat in the different regions, increasing yield is a primary objective in all programs. Grain yield of rice is determined by the number of panicles per unit area, the number of fertile florets per panicle, and grain weight per floret. Increases in any or all of these yield components may provide a mechanism to obtain higher yields. Heritable variation exists for all of these components, and breeders may directly or indirectly select for increases in any of them.

There are numerous steps in the development of any novel, desirable plant germplasm. Plant breeding begins with the analysis and definition of problems and weaknesses of the current germplasm, the establishment of program goals, and the definition of specific breeding objectives. The next step is selection of germplasm that possess the traits to meet the program goals. The goal is to combine in a single variety an improved combination of desirable traits from the parental germplasm. These important traits may include higher seed yield, resistance to diseases and insects, better stems and roots, resistance to low temperatures, resistance to herbicides, and better agronomic characteristics on grain quality.

Choice of breeding or selection methods depends on the mode of plant reproduction, the heritability of the trait(s) being improved, and the type of cultivar used commercially (e.g., F1 hybrid cultivar, pureline cultivar, etc.). For highly heritable traits, a choice of superior individual plants evaluated at a single location will be effective, whereas for traits with low heritability, selection should be based on mean values obtained from replicated evaluations of families of related plants. Popular selection methods commonly include pedigree selection, modified pedigree selection, mass selection, and recurrent selection, or a combination of these methods.

The complexity of inheritance influences choice of the breeding method. Backcross breeding is used to transfer one or a few favorable genes for a highly heritable trait into a desirable cultivar. This approach has been used extensively for breeding disease-resistant cultivars. Various recurrent selection techniques are used to improve quantitatively inherited traits controlled by numerous genes. The use of recurrent selection in self-pollinating crops depends on the ease of pollination, the frequency of successful hybrids from each pollination, and the number of hybrid offspring from each successful cross.

Each breeding program should include a periodic, objective evaluation of the efficiency of the breeding procedure. Evaluation criteria vary depending on the goal and objectives, but should include gain from selection per year based on comparisons to an appropriate standard, overall value of the advanced breeding lines, and number of successful cultivars produced per unit of input (e.g., per year, per dollar expended, etc.).

Promising advanced breeding lines are thoroughly tested and compared to appropriate standards in environments representative of the commercial target area(s) for three or more years. The best lines are candidates for new commercial cultivars; those still deficient in a few traits may be used as parents to produce new populations for further selection.

These processes, which lead to the final step of marketing and distribution, usually take from 8 to 12 years from the time the first cross is made and may rely on the development of improved breeding lines as precursors. Therefore, development of new cultivars is a time-consuming process that requires precise forward planning, efficient use of resources, and a minimum of changes in direction.

A most difficult task is the identification of individuals that are genetically superior, because for most traits the true genotypic value is masked by other confounding plant traits or environmental factors. One method of identifying a superior plant is to observe its performance relative to other experimental plants and to a widely grown standard cultivar. If a single observation is inconclusive, replicated observations provide a better estimate of its genetic worth.

The goal of rice plant breeding is to develop new, unique and superior rice cultivars and hybrids. The breeder initially selects and crosses two or more parental lines, followed by self-pollination and selection, producing many new genetic combinations. The breeder can theoretically generate billions of different genetic combinations via crossing, selfing and mutations. The breeder has no direct control at the cellular level. Therefore, two breeders will never develop the same line, or even very similar lines, having the same rice traits.

Each year, the plant breeder selects the germplasm to advance to the next generation. This germplasm is grown under unique and different geographical, climatic and soil conditions, and further selections are then made, during and at the end of the growing season. The cultivars which are developed are unpredictable. This unpredictability is because the breeder's selection occurs in unique environments, with no control at the DNA level (using conventional breeding procedures), and with millions of different possible genetic combinations being generated. A breeder of ordinary skill in the art cannot predict the final resulting lines he develops, except possibly in a very gross and general fashion. The same breeder cannot produce the same cultivar twice by using the exact same original parents and the same selection techniques. This unpredictability results in the expenditure of large amounts of research monies to develop superior new rice cultivars.

The development of new rice cultivars requires the development and selection of rice varieties, the crossing of these varieties and selection of superior hybrid crosses. The hybrid seed is produced by manual crosses between selected male-fertile parents or by using male sterility systems. These hybrids are selected for certain single gene traits such as semi-dwarf plant type, pubescence, awns, and apiculus color which indicate that the seed is truly a hybrid. Additional data on parental lines, as well as the phenotype of the hybrid, influence the breeder's decision whether to continue with the specific hybrid cross.

Pedigree breeding and recurrent selection breeding methods are used to develop cultivars from breeding populations. Breeding programs combine desirable traits from two or more cultivars or various broad-based sources into breeding pools from which cultivars are developed by selfing and selection of desired phenotypes. The new cultivars are evaluated to determine which have commercial potential.

Pedigree breeding is used commonly for the improvement of self-pollinating crops. Two parents which possess favorable, complementary traits are crossed to produce an F₁. An F₂ population is produced by selfing one or several F₁'s. Selection of the best individuals may begin in the F₂ population; then, beginning in the F₃, the best individuals in the best families are selected. Replicated testing of families can begin in the F₄ generation to improve the effectiveness of selection for traits with low heritability. At an advanced stage of inbreeding (i.e., F₆ and F₇), the best lines or mixtures of phenotypically similar lines are tested for potential release as new cultivars.

Mass and recurrent selections can be used to improve populations of either self- or cross-pollinating crops. A genetically variable population of heterozygous individuals is either identified or created by intercrossing several different parents. The best plants are selected based on individual superiority, outstanding progeny, or excellent combining ability. The selected plants are intercrossed to produce a new population in which further cycles of selection are continued.

Backcross breeding has been used to transfer genes for a simply inherited, highly heritable trait into a desirable homozygous cultivar or inbred line which is the recurrent parent. The source of the trait to be transferred is called the donor parent. The resulting plant is expected to have the attributes of the recurrent parent (e.g., cultivar) and the desirable trait transferred from the donor parent. After the initial cross, individuals possessing the phenotype of the donor parent are selected and repeatedly crossed (backcrossed) to the recurrent parent. The resulting plant is expected to have the attributes of the recurrent parent (e.g., cultivar) and the desirable trait transferred from the donor parent.

The single-seed descent procedure in the strict sense refers to planting a segregating population, harvesting a sample of one seed per plant, and using the one-seed sample to plant the next generation. When the population has been advanced from the F₂ to the desired level of inbreeding, the plants from which lines are derived will each trace to different F₂ individuals. The number of plants in a population declines each generation due to failure of some seeds to germinate or some plants to produce at least one seed. As a result, not all of the F₂ plants originally sampled in the population will be represented by a progeny when generation advance is completed.

In a multiple-seed procedure, rice breeders commonly harvest one or more seeds from each plant in a population and thresh them together to form a bulk. Part of the bulk is used to plant the next generation and part is put in reserve. The procedure has been referred to as modified single-seed descent or the pod-bulk technique.

The multiple-seed procedure has been used to save labor at harvest. It is considerably faster to thresh panicles with a machine than to remove one seed from each by hand for the single-seed procedure. The multiple-seed procedure also makes it possible to plant the same number of seeds of a population each generation of inbreeding. Enough seeds are harvested to make up for those plants that did not germinate or produce seed.

Mutation breeding is another method of introducing new traits into rice lines. Mutations that occur spontaneously or are artificially induced can be useful sources of variability for a plant breeder. The goal of artificial mutagenesis is to increase the rate of mutation for a desired characteristic. Mutation rates can be increased by many different means including temperature, long-term seed storage, tissue culture conditions, radiation (such as X-rays, Gamma rays, neutrons, Beta radiation, or ultraviolet radiation), chemical mutagens (such as base analogs like 5-bromo-uracil), alkylating agents (such as sulfur mustards, nitrogen mustards, epoxides, ethyleneamines, sulfates, sulfonates, sulfones, or lactones), azide, hydroxylamine, nitrous acid or acridines. Once a desired phenotype is observed the genetic mutation responsible for that trait may then be incorporated into existing germplasm by traditional breeding techniques. Details of mutation breeding can be found in Principles of Cultivar Development by Fehr, Macmillan Publishing Company, 1993.

Descriptions of other breeding methods that are commonly used for different traits and crops can be found in one of several reference books (e.g., Allard, 1960; Simmonds, 1979; Sneep et al., 1979; Fehr, 1987).

### Genetic Analysis

In addition to phenotypic observations, the genotype of a plant can also be examined. There are many laboratory-based techniques available for the analysis, comparison and characterization of plant genotype; among these are Isozyme Electrophoresis, Restriction Fragment Length Polymorphisms (RFLPs), Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length polymorphisms (AFLPs), Simple Sequence Repeats (SSRs—which are also referred to as Microsatellites), and Single Nucleotide Polymorphisms (SNPs).

Isozyme Electrophoresis and RFLPs have been widely used to determine genetic composition. Shoemaker and Olsen (Molecular Linkage Map of Soybean (Glycine max), pp. 6.131-6.138 in S. J. O'Brien (ed.) Genetic Maps: Locus Maps of Complex Genomes, Cold Spring Harbor Laboratory Press, Cold Spring Harbor, N.Y. (1993)) developed a molecular genetic linkage map that consisted of 25 linkage groups with about 365 RFLP, 11 RAPD, three classical markers, and four isozyme loci. See also, Shoemaker, R. C., RFLP Map of Soybean, pp. 299-309, in Phillips, R. L. and Vasil, I. K. (eds.), DNA-Based Markers in Plants, Kluwer Academic Press, Dordrecht, the Netherlands (1994).

The invention further provides a method of determining the genotype of a rice plant having oxyfluorfen resistance and containing mutant allele ROXY, or a first generation progeny thereof, which may comprise obtaining a sample of nucleic acids from said plant and detecting in said nucleic acids a plurality of polymorphisms. This method may additionally comprise the step of storing the results of detecting the plurality of polymorphisms on a computer readable medium. The plurality of polymorphisms are indicative of and/or give rise to the expression of the morphological and physiological characteristics of a rice plant containing mutant allele ROXY.

With any of the genotyping techniques mentioned herein, polymorphisms may be detected when the genotype and/or sequence of the plant of interest is compared to the genotype and/or sequence of one or more reference plants. The polymorphism revealed by these techniques may be used to establish links between genotype and phenotype. The polymorphisms may thus be used to predict or identify certain phenotypic characteristics, individuals, or even species. The polymorphisms are generally called markers. It is common practice for the skilled artisan to apply molecular DNA techniques for generating polymorphisms and creating markers. The polymorphisms of this invention may be provided in a variety of mediums to facilitate use, e.g. a database or computer readable medium, which may also contain descriptive annotations in a form that allows a skilled artisan to examine or query the polymorphisms and obtain useful information.

SSR technology is currently the most efficient and practical marker technology; more marker loci can be routinely used and more alleles per marker locus can be found using SSRs in comparison to RFLPs. Gealy, David, et al. (2005) “Insights into the Parentage of Rice/red Rice Crosses Using SSR Analysis of US Rice Cultivars and Red Rice Populations”. Rice Technical Working Group Meeting Proceedings. Abstract p. 179; Lawson, Mark J., et al. (2006) “Distinct Patterns of SSR Distribution in the Arabidopsis thaliana and rice genomes” Genome Biology. 7:R14; Nagaraju, J., et al., (2002) “Genetic Analysis of Traditional and Evolved Basmati and Non-Basmati Rice Varieties by Using Fluorescence-based ISSR-PCR and SSR Markers” Proc. Nat. Acad. Sci. USA. 99(9):5836-5841; and Lu, Hong, et al. (2005) “Population Structure and Breeding Patterns of 145 US Rice Cultivars Based on SSR Marker Analysis” Crop Science. 45:66-76. Single Nucleotide Polymorphisms (SNPs) may also be used to identify the unique genetic composition of the invention and progeny varieties retaining that unique genetic composition. Various molecular marker techniques may be used in combination to enhance overall resolution.

Molecular markers, which include markers identified through the use of techniques such as Isozyme Electrophoresis, RFLPs, RAPDs, AP-PCR, DAF, SCARs, AFLPs, SSRs, and SNPs, may be used in plant breeding. One use of molecular markers is Quantitative Trait Loci (QTL) mapping. QTL mapping is the use of markers which are known to be closely linked to alleles that have measurable effects on a quantitative trait. Selection in the breeding process is based upon the accumulation of markers linked to the positive effecting alleles and/or the elimination of the markers linked to the negative effecting alleles from the plant's genome.

Molecular markers can also be used during the breeding process for the selection of qualitative traits. For example, markers closely linked to alleles or markers containing sequences within the actual alleles of interest can be used to select plants that contain the alleles of interest during a backcrossing breeding program. The markers can also be used to select toward the genome of the recurrent parent and against the markers of the donor parent. This procedure attempts to minimize the amount of genome from the donor parent that remains in the selected plants. It can also be used to reduce the number of crosses back to the recurrent parent needed in a backcrossing program. The use of molecular markers in the selection process is often called genetic marker enhanced selection or marker-assisted selection. Flanking markers that are tightly linked to target genes can be used for selection and are sometimes more efficient than direct selection for the target genes. Use of flanking markers on either side of the locus of interest during marker assisted selection increases the probability that the desired gene is selected. Molecular markers may also be used to identify and exclude certain sources of germplasm as parental varieties or ancestors of a plant by providing a means of tracking genetic profiles through crosses.

Particular markers used for these purposes are not limited to the set of markers disclosed herein, but may include any type of marker and marker profile which provides a means of distinguishing varieties. In addition to being used for identification of rice plants containing mutant allele ROXY, a hybrid produced through the use of mutant allele ROXY, and the identification or verification of pedigree for progeny plants produced through the use of rice plants containing mutant allele ROXY, a genetic marker profile is also useful in developing a locus conversion of rice plants containing mutant allele ROXY.

Means of performing genetic marker profiles using SNP and SSR polymorphisms are well known in the art. SNPs are genetic markers based on a polymorphism in a single nucleotide. A marker system based on SNPs can be highly informative in linkage analysis relative to other marker systems in that multiple alleles may be present.

Rice DNA molecular marker linkage maps have been rapidly constructed and widely implemented in genetic studies such as in Zhu, J. H., et al. (1999) “Toward rice genome scanning by map-based AFLP fingerprinting” Mol. Gene Genetics. 261(1):184-195; Cheng, Z., et al (2001) “Toward a cytological characterization of the rice genome” Genome Research. 11(12):2133-2141; Ahn, S., et al. (1993) “Comparative linkage maps of the rice and maize genomes” Proc. Natl. Acad. Sci. USA. 90(17):7980-7984; and Kao, F. I., et al. (2006) “An integrated map of Oryza sativa L. chromosome 5” Theor. Appl. Genet. 112(5):891-902. Sequences and PCR conditions of SSR Loci in rice as well as the most current genetic map may be found in RiceBLAST and the TIGR Rice Genome Annotation on the World Wide Web.

Proper testing should detect any major faults and establish the level of superiority or improvement over current cultivars. In addition to showing superior performance, there must be a demand for a new cultivar that is compatible with industry standards or which creates a new market. The introduction of a new cultivar will incur additional costs to the seed producer, the grower, processor and consumer; for special advertising and marketing, altered seed and commercial production practices, and new product utilization. The testing preceding release of a new cultivar should take into consideration research and development costs as well as technical superiority of the final cultivar. For seed-propagated cultivars, it must be feasible to produce seed easily and economically.

Rice varieties containing mutant allele ROXY of the present invention can also be used for transformation where exogenous genes are introduced and expressed by the variety containing mutant allele ROXY. Genetic variants created either through traditional breeding methods using a line containing mutant allele ROXY or through transformation of a line containing mutant allele ROXY by any of a number of protocols known to those of skill in the art are intended to be within the scope of this invention.

The following describes breeding methods that may be used with a rice plant containing mutant allele ROXY in the development of further rice plants. One such embodiment is a method for developing a progeny rice plant in a rice plant breeding program comprising: obtaining a rice plant, or a part thereof, which comprises mutant allele ROXY, utilizing said plant or plant part as a source of breeding material and selecting a progeny plant containing mutant allele ROXY with molecular markers in common with rice plants containing mutant allele ROXY. Breeding steps that may be used in the rice plant breeding program include pedigree breeding, back crossing, mutation breeding, and recurrent selection. In conjunction with these steps, techniques such as RFLP-enhanced selection, genetic marker enhanced selection (for example SSR markers) and the making of double haploids may be utilized. Double haploids are produced by the doubling of a set of chromosomes (1 N) from a heterozygous plant to produce a completely homozygous individual. For example, see Wan et al., “Efficient Production of Doubled Haploid Plants Through Colchicine Treatment of Anther-Derived Maize Callus”, Theoretical and Applied Genetics, 77:889-892, 1989 and U.S. Pat. No. 7,135,615.

One of ordinary skill in the art of plant breeding would know how to evaluate the traits of two plant varieties to determine if there is no significant difference between the two traits expressed by those varieties. For example, see Fehr and Walt, Principles of Cultivar Development, p 261-286 (1987). Thus the invention includes rice plants containing mutant allele ROXY progeny rice plants so that said progeny rice plants are not significantly different for said traits than rice plants containing mutant allele ROXY as determined at the 5% significance level when grown in the same environment. Using techniques described herein, molecular markers may be used to identify said progeny plant as a plant containing mutant allele ROXY progeny plant. Mean trait values may be used to determine whether trait differences are significant, and preferably the traits are measured on plants grown under the same environmental conditions. Once such a variety is developed its value is substantial since it is important to advance the germplasm base as a whole in order to maintain or improve traits such as yield, disease resistance, pest resistance, and plant performance in extreme environmental conditions.

Progeny of rice plants containing mutant allele ROXY may also be characterized through their filial relationship with rice plants containing mutant allele ROXY, as for example, being within a certain number of breeding crosses of rice plants containing mutant allele ROXY. A breeding cross is a cross made to introduce new genetics into the progeny, and is distinguished from a cross, such as a self or a sib cross, made to select among existing genetic alleles. The lower the number of breeding crosses in the pedigree, the closer the relationship between rice plants containing mutant allele ROXY and its progeny. For example, progeny produced by the methods described herein may be within 1, 2, 3, 4 or 5 breeding crosses of rice plants containing mutant allele ROXY.

The seed of rice plants containing mutant allele ROXY, the plant produced from the seed, the hybrid rice plant produced from the crossing of the cultivar, hybrid seed, and various parts of the hybrid rice plant and transgenic versions of the foregoing, can be utilized for human food, livestock feed, and as a raw material in industry.

### EXAMPLES

The following examples are provided to further illustrate the present invention and are not intended to limit the invention beyond the limitations set forth in the appended claims.

### Example 1—Development of Mutant Rice Lines and Mutant Allele ROXY

Seed (3 kg) of the rice cultivar ‘M-206’ (U.S. Pat. No. 6,911,589 to Johnson issued Jun. 28, 2005) was treated with a chemical mutagen, 2% ethyl methane sulfonate, and the M₁ plants were grown in the greenhouse in the winter of 2012-13 and harvested. The M₂ generation was grown in the field and harvested in bulk in the fall of 2013. The resulting M₃ seed was planted on soil in greenhouse benches (1 kg/9.3 m²) and watered to germinate and grow to a seedling height of approximately 20 cm. The seedlings were then sprayed with Goal® 2XL at 2 pt./acre (560 g ai/ha). Unexpectedly, twenty-nine putative resistant seedlings that were not killed by the treatment were recovered. The seedlings were transferred to pots and allowed to grow to maturity and seed harvested. The M₄ seed of the 29 putative oxyfluorfen resistant mutant plants were pre-germinated and placed on saturated clay soil and sprayed with Goal® 2XL at 2 pt./acre (560 g ai/ha) in a spray chamber and allowed to grow in lighted greenhouse benches kept saturated by sub-irrigation. Unexpectedly, seedlings from lines derived from M₃ plants 1 to 9 grew through the herbicide treatment and the others did not survive.

The test was repeated and included the California medium grain rice cultivars M-205 and the parent M-206. Lines from plants 1-9 grew through the herbicide treatment and the other selections and M-205 and M-206 did not survive. Lines from plants 1-9 were designated 14G1, 14G2, 14G3, 14G4, 14G5, 14G6, 14G7, 14G8, and 14G9 and were concluded to have a mutant allele, which was later designated ROXY. The surviving seedlings from the tests were grown to maturity and screened in 2015, and two additional plants were recovered and confirmed in a similar herbicide screening of residual M₃ seed and designated 15G3 and 15G4, also concluded to contain mutant allele ROXY.

### Example 2—Screening Mutant Rice Lines Containing Mutant Allele ROXY

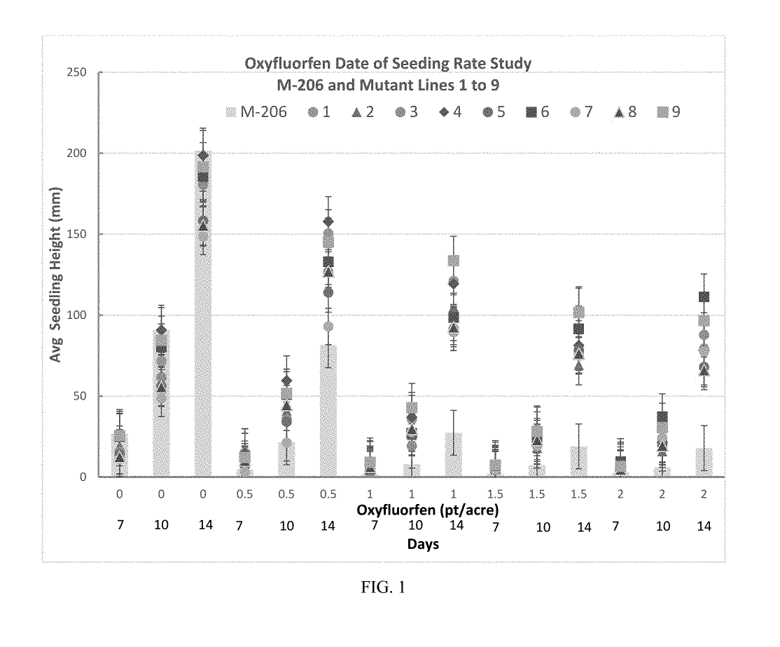

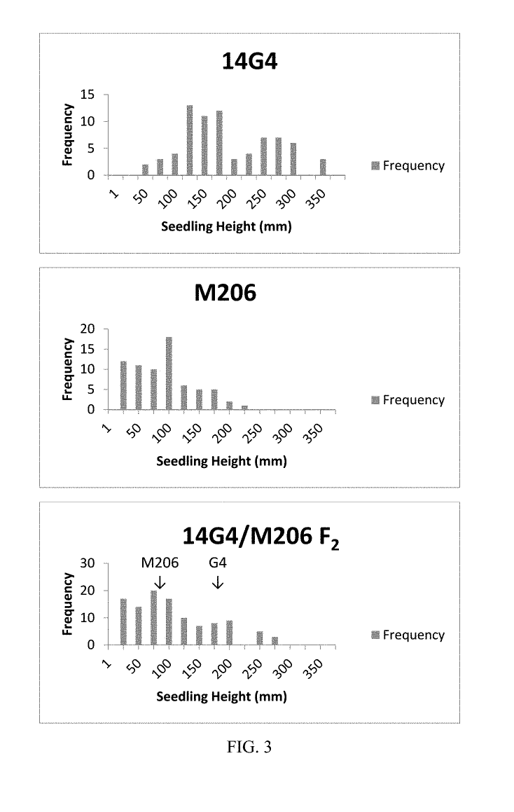

Seeds of lines 14G1, 14G2, 14G3, 14G4, 14G5, 14G6, 14G7, 14G8, and 14G9 and M-206 were pre-germinated and ten seeds in a row were placed on saturated soil in five trays. The trays were sprayed in a spray chamber with 0, 0.5 1.0, 1.5, and 2.0 pt./acre of oxyfluorfen (Goal® 2XL). The trays were placed in benches in a lighted greenhouse and the soil was kept saturated by sub-irrigation. Seedling height was measured at 7, 10, and 14 days after treatment. FIG. 3 shows the improved resistance to oxyfluorfen of lines 14G1, 14G2, 14G3, 14G4, 14G5, 14G6, 14G7, 14G8, and 14G9 containing mutant allele ROXY over M-206 as reflected by the growth of the seedling (average seedling height). Unexpectedly, by the measurement of seedling height at 14 days, rice lines 14G1, 14G2, 14G3, 14G4, 14G5, 14G6, 14G7, 14G8, and 14G9 containing mutant allele ROXY were significantly taller than M-206 at the 1 pt./acre rate (280 g ai/ha) or higher, as shown in FIG. 1.

### Example 3—Field Testing of Mutant Rice Lines Containing Mutant Allele ROXY

Seed of the oxyfluorfen resistant lines 14G1, 14G2, 14G3, 14G4, 14G5, 14G6, 14G7, 14G8, and 14G9 containing mutant allele ROXY were increased in the greenhouse and provided seed for a small plot field test at the nursery at Biggs, Calif. in 2015. The experiment included rice lines 14G1, 14G2, 14G3, 14G4, 14G5, 14G6, 14G7, 14G8, and 14G9 containing mutant allele ROXY and M-206 without mutant allele ROXY in 4×6 foot water-seeded plots with two replications. Goal® 2XL at 2 pt./acre (560 g ai/ha) was sprayed onto the water immediately after seeding. Unexpectedly, lines 14G1, 14G2, 14G3, 14G4, 14G5, 14G6, 14G7, 14G8, and 14G9 containing mutant allele ROXY emerged through the water, whereas the M-206 without mutant allele ROXY was slow in emerging as reflected in the low seedling vigor score. M-206 seedling survival was low, resulting in very few plants in the plot (FIG. 2). The few plants in the plots of M-206 resulted in low grain produced per plot, averaging significantly less than the oxyfluorfen resistant lines 14G1, 14G2, 14G3, 14G4, 14G5, 14G6, 14G7, 14G8, and 14G9 containing mutant allele ROXY as summarized in Table 1. Table 1, column 1 shows the rice line, column 2 shows the replication, column 3 shows the seedling vigor (SV) score of 1 to 5 where 1 indicates poor and 5 indicates good, column 4 shows the days to 50% heading, column 5 shows the plant height in centimeters (cm), column 6 shows the percent lodging and column 7 shows the plot yield in grams (g).

TABLE 1

| Line | | SV | Heading | Height | Lodging | Yield |
|---|---|---|---|---|---|---|
| ID | Replication | score | (days) | (cm) | (%) | (g) |
| 14G1 | 1 | 4.0 | 76 | 90 | 20 | 3084 |
| | 2 | 4.0 | 76 | 95 | 20 | 3420 |
| | avg | 4.0 | 76 | 93 | 20 | 3252 |
| 14G2 | 1 | 4.0 | 76 | 88 | 20 | 2953 |
| | 2 | 4.5 | 77 | 95 | 20 | 3294 |
| | avg | 4.3 | 77 | 92 | 20 | 3123 |
| 14G3 | 1 | 4.0 | 77 | 92 | 30 | 2832 |
| | 2 | 4.5 | 76 | 94 | 30 | 3350 |
| | avg | 4.3 | 77 | 93 | 30 | 3091 |
| 14G4 | 1 | 4.5 | 76 | 95 | 20 | 3228 |
| | 2 | 4.0 | 76 | 95 | 30 | 3073 |
| | avg | 4.3 | 76 | 95 | 25 | 3151 |
| 14G5 | 1 | 4.0 | 76 | 90 | 20 | 2728 |
| | 2 | 4.0 | 76 | 91 | 20 | 2800 |
| | avg | 4.0 | 76 | 91 | 20 | 2764 |
| 14G6 | 1 | 4.0 | 78 | 87 | 20 | 2796 |
| | 2 | 4.0 | 78 | 90 | 10 | 2639 |
| | avg | 4.0 | 78 | 89 | 15 | 2718 |
| 14G7 | 1 | 4.0 | 76 | 92 | 20 | 2448 |
| | 2 | 4.0 | 76 | 95 | 30 | 2836 |
| | avg | 4.0 | 76 | 94 | 25 | 2642 |
| 14G8 | 1 | 3.5 | 77 | 91 | 10 | 1926 |
| | 2 | 3.0 | 77 | 96 | 20 | 2813 |
| | avg | 3.3 | 77 | 94 | 15 | 2369 |
| 14G9 | 1 | 4.5 | 76 | 93 | 30 | 3167 |
| | 2 | 4.7 | 77 | 100 | 40 | 4325 |
| | avg | 4.6 | 77 | 97 | 35 | 3746 |
| M206 | 1 | 0.5 | 77 | 85 | 10 | 800 |
| | 2 | 0.5 | 77 | 85 | 10 | 1312 |
| | avg | 0.5 | 77 | 85 | 10 | 1056 |
| LSD | | 0.5 | 0.9 | 4.2 | 11 | 668 |

### Example 4—Water Seeded Testing of Mutant Rice Lines Containing Mutant Allele ROXY

Seed of the oxyfluorfen resistant rice lines 14G1, 14G2, 14G3, 14G4, 14G5, 14G6, 14G7, 14G8, and 14G9 containing mutant allele ROXY were increased in the greenhouse and provided seed for water seeded testing of lines 14G1, 14G2, 14G3, 14G4, 14G5, 14G6, 14G7, 14G8, and 14G9 containing mutant allele ROXY and M-206 planted in single five foot rows in separate basins that received seven different treatments of oxyfluorfen (Goal® 2XL) at the nursery at Biggs, Calif. in 2015. The treatments included no oxyfluorfen, preflood treatment with oxyfluorfen at 1 pt./acre, date of seeding treatment with oxyfluorfen at 1 pt./acre, second leaf stage treatment with oxyfluorfen at 1 pt./acre, preflood treatment with oxyfluorfen at 2 pt./acre, date of seeding treatment with oxyfluorfen at 2 pt./acre, and second leaf stage treatment with oxyfluorfen at 2 pt./acre. Table 2 shows the average values for oxyfluorfen resistant rice lines 14G1, 14G2, 14G3, 14G4, 14G5, 14G6, 14G7, 14G8, and 14G9 containing mutant allele ROXY compared to rice line M-206 without mutant allele ROXY. Table 2, column 1 shows the rice line, column 2 shows the days to 50% heading, column 3 shows the height in centimeters (cm), and column 4 shows the grain weight in grams (g). As shown in Table 2, all rice lines containing mutant allele ROXY produced more grain than rice line M-206 indicating their improved resistance to oxyfluorfen applications.

Table 3 show the average values for all the rows in each treatment. The lower grain production in the check is due to weed competition that was not present in the other treatments, indicating that the oxyfluorfen was providing weed control in all treatments. In Table 3, column 1 shows the treatment, column 2 shows the number of days to 50% heading, column 3 shows the height in centimeters (cm) and column 4 shows the grain weight in grams (g). In Table 3, the oxyfluorfen (Goal® 2XL) treatment stages are abbreviated as follows: CK=no Goal® 2XL applied, PP1=preflood 1 pt./acre. DOS1=date of seeding 1 pt./acre, 2ndlf1=2^{nd} leaf stage1 pt./acre, PP2=preflood 2 pt./acre. DOS2=date of seeding 2 pt./acre and 2ndlf2=2^{nd} leaf stage2 pt./acre.

TABLE 2

| Line | Heading | Height | Grain |
|---|---|---|---|
| ID | (days) | (cm) | (g) |
| 14G1 | 71 | 91 | 501 |
| 14G2 | 72 | 91 | 466 |
| 14G3 | 72 | 94 | 595 |
| 14G4 | 72 | 96 | 527 |
| 14G5 | 72 | 92 | 412 |
| 14G6 | 72 | 93 | 557 |
| 14G7 | 72 | 94 | 414 |
| 14G8 | 72 | 97 | 488 |
| 14G9 | 72 | 96 | 586 |
| M206 | 73 | 95 | 293 |

TABLE 3

| | Heading | Height | Grain |
|---|---|---|---|
| Treatment | (days) | (cm) | (g) |
| CK | 70 | 91 | 287 |
| PP1 | 71 | 95 | 456 |
| DOS1 | 73 | 94 | 412 |
| 2ndlf1 | 70 | 94 | 317 |
| PP2 | 72 | 95 | 657 |
| DOS2 | 73 | 94 | 562 |
| 2ndlf2 | 73 | 91 | 500 |

### Example 5—Water Seeded Testing of Mutant Rice Lines Containing Mutant Allele ROXY in a Commercial Rice Field

Seed of the oxyfluorfen resistant rice lines 14G1, 14G2, 14G3, 14G4, 14G5, 14G6, 14G7, 14G8, and 14G9 containing mutant allele ROXY were increased in the greenhouse and provided seed for water seeded tests of lines 14G1, 14G2, 14G3, 14G4, 14G5, 14G6, 14G7, 14G8, and 14G9 containing mutant allele ROXY and M-206 without mutant allele ROXY in a commercial rice field in Glenn County, Calif. in 2015. The experiment included nine treatment basins with different application timings and two rates of oxyfluorfen (GoalTender®). Single 2.5 ft. rows of rice lines 14G1, 14G2, 14G3, 14G4, 14G5, 14G6, 14G7, 14G8, and 14G9 and M-206 were water seeded in a spoke pattern in 5 ft. diameter round metal rings. The rings were covered to allow commercial aerial seeding with a commercial rice variety. The covers were removed after seeding and the rings were removed for spray treatments and replaced, and finally removed after the seedlings had germinated and anchored themselves. The treatments included: no oxyfluorfen, preflood, 1^{st} leaf, 2^{nd} leaf, and 3^{rd} leaf growth stages for both 1 and 2 pt./acre of GoalTender®, 560 and 1121 g ai/ha, respectively.

Table 4 shows the seedling vigor, heading and height for oxyfluorfen resistant rice lines 14G1, 14G2, 14G3, 14G4, 14G5, 14G6, 14G7, 14G8, and 14G9 containing mutant allele ROXY compared to M-206 without mutant allele ROXY. In all but the untreated control applications, the seedling vigor of M-206 was less that rice lines 14G1, 14G2, 14G3, 14G4, 14G5, 14G6, 14G7, 14G8, and 14G9 containing mutant allele ROXY, a reflection of the higher resistance to the herbicide for the mutant lines. Days to heading for M-206 was generally later than rice lines 14G1, 14G2, 14G3, 14G4, 14G5, 14G6, 14G7, 14G8, and 14G9 containing mutant allele ROXY at the higher oxyfluorfen treatment, reflecting the sensitivity and delay caused by the herbicide treatment.

Grain was harvested from each ring (different treatments) at maturity and the weight is shown in Table 4. The control and the preflood treatments all produced a similar amount of grain, whereas the others were somewhat lower. The most complete weed control was provided by the preflood treatment at 2 pt./acre. Table 4, column 1 shows the row number, column 2 shows the rice line, column 3 shows the seedling vigor (SV) score of 1 to 5 where 1 is poor and 5 is good, column 4 shows the days to 50% heading, column 5 shows the height in centimeters (cm), column 6 shows the treatment, where CK=no GoalTender® applied; PP1=preflood−1 pt./acre; 2LSR−1 pt./acre=2^{nd} leaf stage rice−1 pt./acre; PP2=preflood−2 pt./acre; 2LSR−2 pt=2^{nd} leaf stage rice−2 pt./acre; 3LSR−2 pt.=3^{rd} leaf stage rice−2 pt./acre; 1LSR−2 pt.=1^{st} leaf stage rice−2 pt./acre; 3LSR−1 pt./acre=3^{rd} leaf stage rice−1 pt./acre; and 1LSR−1 pt./acre=1^{st} leaf stage rice−1 pt./acre, column 6 shows the grain weight in grams per ring (g/ring), column 7 shows the moisture percent, and column 8 shows the visual weed control score for rice field bulrush (RFB) Schoenoplectus mucronatus, small flower umbrella (SF) Cypres difformis and ducksalad (DS) Heteranthera limosa and H. rotundifolia from 1 to 5 where 0=no control and 5=complete control. Oxyflurofen GoalTender® herbicide treatments gave excellent control of the aforementioned rice weeds in the experiment, especially in the preplant applications.

TABLE 4

| | | | | | | | | Weed Control | | |
|---|---|---|---|---|---|---|---|---|---|---|
| | Rice | SV | Heading | Height | | Grain | Moisture | Score | | |
| Row # | Line | (1-5) | (days) | (cm) | Treatment | (g/ring) | (%) | RFB | SF | DS |
| 60701 | M206 | 5 | 78 | 94 | CK | | | | | |
| 60702 | 14G4 | 5 | 78 | 94 | CK | | | | | |
| 60703 | 14G2 | 5 | 78 | 94 | CK | | | | | |
| 60704 | 14G7 | 5 | 78 | 94 | CK | | | | | |
| 60705 | 14G9 | 5 | 78 | 94 | CK | | | | | |
| 60706 | 14G3 | 5 | 78 | 94 | CK | | | | | |
| 60707 | 14G8 | 5 | 78 | 94 | CK | | | | | |
| 60708 | 14G1 | 5 | 78 | 94 | CK | | | | | |
| 60709 | 14G5 | 5 | 78 | 94 | CK | | | | | |
| 60710 | 14G6 | 5 | 78 | 94 | CK | | | | | |
| Avg | | 5 | 78 | 94 | | 2022 | 19.4 | 0 | 0 | 0 |
| 60711 | 14G9 | 5 | 82 | 96 | PP1 | | | | | |
| 60712 | 14G6 | 5 | 82 | 96 | PP1 | | | | | |
| 60713 | 14G1 | 5 | 82 | 96 | PP1 | | | | | |
| 60714 | 14G5 | 5 | 81 | 96 | PP1 | | | | | |
| 60715 | 14G3 | 5 | 82 | 96 | PP1 | | | | | |
| 60716 | M206 | 2 | 82 | 96 | PP1 | | | | | |
| 60717 | 14G4 | 5 | 82 | 96 | PP1 | | | | | |
| 60718 | 14G7 | 5 | 81 | 96 | PP1 | | | | | |
| 60719 | 14G8 | 5 | 82 | 96 | PP1 | | | | | |
| 60720 | 14G2 | 5 | 83 | 96 | PP1 | | | | | |
| Avg | | 4.7 | 81.9 | 96 | | 2014 | 16.1 | 3 | 5 | 5 |
| 60721 | 14G9 | 2 | 80 | 93 | 2LSR-1pt | | | | | |
| 60722 | 14G6 | 3 | 80 | 93 | 2LSR-1pt | | | | | |
| 60723 | 14G1 | 4 | 80 | 93 | 2LSR-1pt | | | | | |
| 60724 | 14G5 | 2.5 | 80 | 93 | 2LSR-1pt | | | | | |
| 60725 | 14G3 | 3 | 80 | 93 | 2LSR-1pt | | | | | |
| 60726 | M206 | 1.5 | 83 | 93 | 2LSR-1pt | | | | | |
| 60727 | 14G4 | 3 | 80 | 93 | 2LSR-1pt | | | | | |
| 60728 | 14G7 | 2.5 | 80 | 93 | 2LSR-1pt | | | | | |
| 60729 | 14G8 | 2.5 | 80 | 93 | 2LSR-1pt | | | | | |
| 60730 | 14G2 | 2 | 80 | 93 | 2LSR-1pt | | | | | |
| Avg | | 2.6 | 80.3 | 93 | | 1384 | 18.8 | 4 | 4 | 1 |
| 60731 | 14G9 | 2.5 | 83 | 93 | PP2 | | | | | |
| 60732 | 14G6 | 2.5 | 83 | 93 | PP2 | | | | | |
| 60733 | 14G1 | 2.5 | 82 | 93 | PP2 | | | | | |
| 60734 | 14G5 | 2.5 | 83 | 93 | PP2 | | | | | |
| 60735 | 14G3 | 2.5 | 82 | 93 | PP2 | | | | | |
| 60736 | M206 | 0.5 | 84 | 93 | PP2 | | | | | |
| 60737 | 14G4 | 2.5 | 81 | 93 | PP2 | | | | | |
| 60738 | 14G7 | 2.5 | 82 | 93 | PP2 | | | | | |
| 60739 | 14G8 | 2.5 | 82 | 93 | PP2 | | | | | |
| 60740 | 14G2 | 2.5 | 82 | 93 | PP2 | | | | | |
| Avg | | 2.3 | 82.4 | 93 | | 2046 | 18.4 | 5 | 5 | 5 |
| 60741 | 14G7 | 0.5 | 83 | 85 | 2LSR-2pt | | | | | |
| 60742 | 14G6 | 0.5 | 84 | 85 | 2LSR-2pt | | | | | |
| 60743 | 14G4 | 0.5 | 82 | 85 | 2LSR-2pt | | | | | |
| 60744 | 14G1 | 0.5 | 82 | 85 | 2LSR-2pt | | | | | |
| 60745 | 14G2 | 0.5 | 84 | 85 | 2LSR-2pt | | | | | |
| 60746 | 14G9 | 0.5 | 82 | 85 | 2LSR-2pt | | | | | |
| 60747 | 14G3 | 0.5 | 83 | 85 | 2LSR-2pt | | | | | |
| 60748 | 14G8 | 0.5 | 83 | 85 | 2LSR-2pt | | | | | |
| 60749 | M206 | 0 | 85 | 85 | 2LSR-2pt | | | | | |
| 60750 | 14G5 | 0.5 | 83 | 85 | 2LSR-2pt | | | | | |
| Avg | | 0.45 | 83.1 | 85 | | 1667 | 16.6 | 5 | 5 | 4 |
| 60751 | 14G7 | 0.5 | 82 | 82 | 3LSR-2pt | | | | | |
| 60752 | 14G2 | 0.5 | 83 | 82 | 3LSR-2pt | | | | | |
| 60753 | 14G4 | 0.5 | 84 | 82 | 3LSR-2pt | | | | | |
| 60754 | 14G8 | 0.5 | 85 | 82 | 3LSR-2pt | | | | | |
| 60755 | M206 | 0 | 86 | 82 | 3LSR-2pt | | | | | |
| 60756 | 14G9 | 0.5 | 83 | 82 | 3LSR-2pt | | | | | |
| 60757 | 14G1 | 0.5 | 83 | 82 | 3LSR-2pt | | | | | |
| 60758 | 14G3 | 0.5 | 83 | 82 | 3LSR-2pt | | | | | |
| 60759 | 14G6 | 1 | 81 | 82 | 3LSR-2pt | | | | | |
| 60760 | 14G5 | 1 | 81 | 82 | 3LSR-2pt | | | | | |
| Avg | | 0.55 | 83.1 | 82 | | 1794 | 20.5 | 5 | 5 | 4 |
| 60761 | 14G5 | 2 | 82 | 95 | 1LSR-2pt | | | | | |
| 60762 | 14G7 | 2 | 80 | 95 | 1LSR-2pt | | | | | |
| 60763 | 14G1 | 2 | 80 | 95 | 1LSR-2pt | | | | | |
| 60764 | 14G9 | 2 | 81 | 95 | 1LSR-2pt | | | | | |
| 60765 | 14G3 | 2 | 81 | 95 | 1LSR-2pt | | | | | |
| 60766 | 14G6 | 2 | 82 | 95 | 1LSR-2pt | | | | | |
| 60767 | 14G4 | 2 | 80 | 95 | 1LSR-2pt | | | | | |
| 60768 | M206 | 1 | 81 | 95 | 1LSR-2pt | | | | | |
| 60769 | 14G2 | 2 | 82 | 95 | 1LSR-2pt | | | | | |
| 60770 | 14G8 | 2 | 80 | 95 | 1LSR-2pt | | | | | |
| Avg | | 1.9 | 80.9 | 95 | | 2480 | 19.5 | 5 | 5 | 3 |
| 60771 | 14G6 | 2.5 | 80 | 93 | 3LSR-1pt | | | | | |
| 60772 | 14G8 | 2.5 | 80 | 93 | 3LSR-1pt | | | | | |
| 60773 | 14G2 | 2.5 | 81 | 93 | 3LSR-1pt | | | | | |
| 60774 | 14G7 | 2.5 | 80 | 93 | 3LSR-1pt | | | | | |
| 60775 | 14G5 | 2.5 | 80 | 93 | 3LSR-1pt | | | | | |
| 60776 | 14G3 | 2.5 | 80 | 93 | 3LSR-1pt | | | | | |
| 60777 | 14G4 | 2.5 | 80 | 93 | 3LSR-1pt | | | | | |
| 60778 | 14G9 | 2.5 | 80 | 93 | 3LSR-1pt | | | | | |
| 60779 | 14G1 | 2.5 | 80 | 93 | 3LSR-1pt | | | | | |
| 60780 | M206 | 0.5 | 84 | 93 | 3LSR-1pt | | | | | |
| Avg | | 2.3 | 80.5 | 93 | | 1794 | 20.5 | 4 | 4 | 1 |
| 60781 | 14G6 | 2.5 | 81 | 91 | 1LSR-1pt | | | | | |
| 60782 | 14G3 | 2.5 | 80 | 91 | 1LSR-1pt | | | | | |
| 60783 | 14G4 | 2.5 | 80 | 91 | 1LSR-1pt | | | | | |
| 60784 | M206 | 0.5 | 81 | 91 | 1LSR-1pt | | | | | |
| 60785 | 14G1 | 2 | 80 | 91 | 1LSR-1pt | | | | | |
| 60786 | 14G8 | 2.5 | 80 | 91 | 1LSR-1pt | | | | | |
| 60787 | 14G2 | 1.5 | 81 | 91 | 1LSR-1pt | | | | | |
| 60788 | 14G5 | 3 | 79 | 91 | 1LSR-1pt | | | | | |
| 60789 | 14G7 | 2.5 | 80 | 91 | 1LSR-1pt | | | | | |
| 60790 | 14G9 | 3 | 79 | 91 | 1LSR-1pt | | | | | |
| Avg | | 2.25 | 80.1 | 91 | | 2006 | 21.0 | 4 | 4 | 2 |

### Example 6—Transferring Mutant Allele ROXY to Different Genetic Backgrounds and Mode of Inheritance

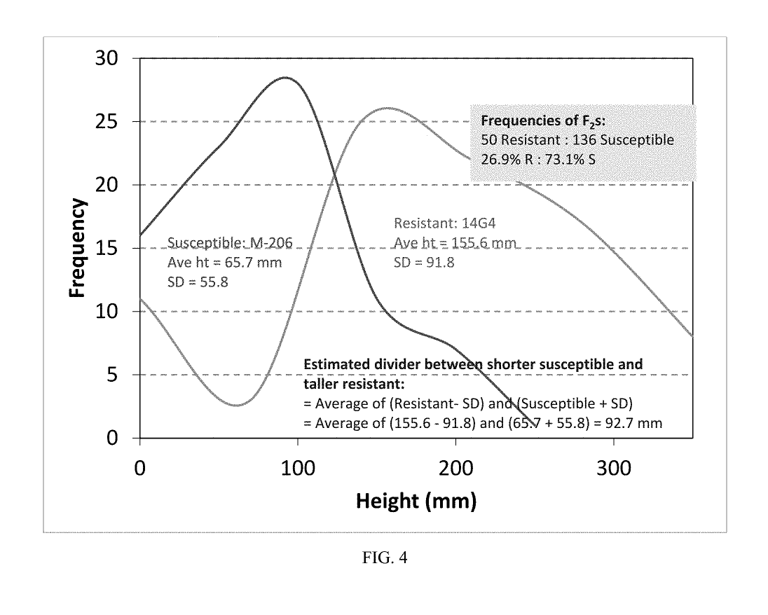

Oxyfluorfen resistant rice line 14G4 containing mutant allele ROXY was crossed with rice line M-206, which does not contain mutant allele ROXY. F₂ seeds from the cross 14G4×M-206 and the parent lines were pre-germinated and space planted on saturated soil in trays. The trays were sprayed in a spray chamber with 2.0 pt./acre (560 g ai/ha) of oxyfluorfen (Goal® 2XL) and were placed in benches in a lighted greenhouse, with the soil kept saturated by sub-irrigation. Seedling height was measured at 14 days after treatment. FIG. 3 shows the characteristic single gene bimodal frequency distribution for the F₂ population and the distribution of the parents for plant seedling height in millimeters (mm). Phenotypic classification of these F₂ plants for short (susceptible) or tall (oxyfluorfen resistance) is shown in FIG. 4. Table 5 shows the good fit to a 3:1 ratio indicating mutant allele ROXY and the oxyfluorfen resistance trait appears to be inherited as a single recessive gene.

TABLE 5

| | | | X² for 3:1 |
|---|---|---|---|
| Phenotype | Observed | Theoretical | inheritance |
| Susceptible | 136 | 139 | =0.25 |
| Resistant | 50 | 47 | 0.50 < P < 0.70 |

The long grain aromatic cultivar A-202 (U.S. Pat. No. 9,338,992 to Jodari et al. issued May 17, 2016), which does not contain mutant allele ROXY, was crossed with the oxyfluorfen resistant rice line 14G7 containing mutant allele ROXY. Ten day old seedlings of F₃ progeny rows from random F₂ plants from the cross A-202×14G7 and the parent lines were sprayed in a spray chamber with 2.0 pt./acre (1121 g ai/ha) of oxyfluorfen (GoalTender®). Plants were allowed to grow in the greenhouse for 10 days and the treatment was repeated and the resistant rows identified. Table 6 shows a fit to a 3:1 ratio indicating mutant allele ROXY and the oxyfluorfen resistance trait appears to be inherited as a single recessive gene and was transferred to a rice in a cross to a more diverse genetic background.

TABLE 6

| | | | X² for 3:1 |
|---|---|---|---|
| Phenotype | Observed | Theoretical | inheritance |
| Susceptible | 116 | 124 | =1.94 |
| Resistant | 49 | 41 | 0.20 < P < 0.10 |

Herbicide resistance of F₃ seedlings in segregating F₃ lines from the study described above were also counted and fit the expected 3:1 segregation ratio of a single recessive gene, as shown in Table 7 below.

TABLE 7

| | | | X² for 3:1 |
|---|---|---|---|
| Phenotype | Observed | Theoretical | inheritance |
| Susceptible | 428 | 427 | =2.16 |
| Resistant | 142 | 143 | 0.99 < P < 0.95 |

### Example 7—Confirming that Mutant Allele ROXY is Inherited as a Single, Recessive Gene

To confirm the hypothesis that mutant allele ROXY of the present invention is inherited as a single recessive gene, random 14G4×M-206 F₂ plants from the previous study presented in Table 5 were allowed to self-pollinate, grow to maturity, and seed was harvested from them individually to test oxyfluorfen resistance of their F₃ progeny. F₃ progeny rows were planted on saturated soil in trays and sprayed in a spray chamber with 2.0 pt./acre (1121 g ai/ha) of oxyfluorfen (GoalTender®). Plants were allowed to grow in the greenhouse for 10 days and the treatment was repeated. Rows were visually scored as susceptible (killed), segregating (resistant and killed seedlings), and resistant to the oxyfluorfen treatments. Table 8 below shows the results of the study, which show a good fit to a 1:2:1 ratio characteristic of single recessive gene inheritance, confirming that ROXY is inherited as a single recessive gene.

TABLE 8

| | | | X² for 1:2:1 |
|---|---|---|---|
| Phenotype | Observed | Theoretical | inheritance |
| Susceptible | 25 | 25.25 | =1.91 |
| Segregating | 45 | 50.50 | 0.50 < P < 0.70 |
| Resistant | 31 | 25.25 | |

Herbicide resistance of F₃ seedlings in segregating F₃ lines from the study described above were also counted and fit the expected 3:1 segregation ratio of a single recessive gene, as shown in Table 9 below.

TABLE 9

| | | | X² for 3:1 |
|---|---|---|---|
| Phenotype | Observed | Theoretical | inheritance |
| Susceptible | 324 | 337.5 | =2.16 |
| Resistant | 126 | 112.5 | 0.10 < P < 0.20 |

F₁ seedlings from crosses with mutant allele ROXY rice lines and susceptible lines not having ROXY were sprayed with GoalTender® following the protocol described above. The seedlings from crosses to susceptible lines (M-105, M-205, 12Y3097, M-209) were killed and the mutant line checks (14G6, 14G9, 14G4) survived demonstrating the recessive nature of this trait, as shown in Table 10. In addition, a cross between two mutant lines (14G9×14G4) was not killed by the herbicide supporting idea that the mutant trait is the same in these different lines, all of which were recovered from the same lot of seed. To provide further evidence that the same mutant allele ROXY is present in all mutant lines, crosses were made between all mutant lines (14G1, 14G2, 14G3, 14G4, 14G5, 14G6, 14G7, 14G8, 14G9, 15G3 and 15G4) in a half diallel. Ten F₁ seeds from each cross combination were planted in rows in trays that include a row of the susceptible M-206. Seedlings were sprayed with GoalTender® following the protocol described above. The M-206 row was killed and all the rows of F₁ mutant seedlings were resistant to the two applications of oxyfluorfen. If the oxyfluorfen resistance of the any of the mutant lines was not the same, then the F₁ rows would have been killed by the herbicide application.

TABLE 10

| Pedigree | Reaction to oxyfluorfen |
|---|---|
| M-105 × 14G4 F₁ | Susceptible |
| M-205 × 14G4 F₁ | Susceptible |
| M-205 × 14G9 F₁ | Susceptible |
| 14G6 | Resistant |
| 12Y3097 × 14G9 F₁ | Susceptible |
| 14G9 × 14G4 F₁ | Resistant |
| 12Y3097 | Susceptible |
| 14G9 | Resistant |
| M-105 | Susceptible |
| M-205 | Susceptible |
| M-209 | Susceptible |
| 14G4 | Resistant |

### Example 8—Resistance to Oxyfluorfen and Determining the DNA Sequence and Location of Mutant Allele ROXY

Oxyfluorfen, a member of the diphenyl ether group of peroxidizing herbicides, is photodynamically active and competitively blocks the substrate-binding region of protoporphyrinogen oxidase (PPO or PROTOX). PPO is the last common enzyme in the tetrapyrrole biosynthetic pathway that produces heme and chlorophyll. While the production of chlorophyll, a light-harvesting pigment, is an essential process for all green photosynthetic organisms, heme is an essential cofactor in cytochromes, haemoglobin, oxygenases, peroxidases and catalases. In plants, chlorophyll biosynthesis takes place exclusively in plastids, whereas heme is produced in both plastids and mitochondria. In both organelles, PPO converts protopophyrinogen IX (protogen IX) to protoporphyrin IX (proto IX). Two different nuclear genes, PPX1 and PPX2, encode plastid and mitochondrial PPO isozymes, respectively.

When susceptible plants are treated with PPO inhibitors, such as oxyfluorfen, protogen IX accumulates and moves away from the reaction center in the chloroplast into the cytoplasm, where herbicide-insensitive peroxidase-like enzymes in the plasma membrane convert it to proto IX. Proto IX accumulates in the cytoplasm and, in the presence of light, induces formation of highly reactive singlet oxygen that is damaging to cell membranes, leading to peroxidation of cell constituents such as lipids, proteins, and nucleic acids. Typical symptoms of oxyfluorfen-treated plants include leaf desiccation, veinal necrosis, and leaf deformation. (Patzoldt et. al., 2006, PNAS; Ha et. al., 2003, Plant, Cell and Environment).

PPO inhibitor-resistant rice plants have been developed, for example, by expression of the Arabidopsis, Bacillus subtilis or Myxococcus xanthus PPO genes via targeting the gene into either chloroplast or cytoplasm. Other attempts to develop PPO herbicide-resistant plants include conventional tissue culture methods, expression of modified co-factors of the protoporphyrin IX binding subunit proteins, over-expression of wild-type plant PPO gene, and engineering of P-450 monooxygenases to degrade the PPO inhibitor. (Ha et. al., 2003, Plant, Cell and Environment; Li and Nicholl, 2005, Pest Manag Sci; Nam et. al., 2016, International Journal of Food Science and Technology; Jung et. al., 2004, Plant, Cell and Environment).

Mutant allele ROXY of the present invention confers resistance to the herbicide oxyfluorfen in rice. The mutant rice lines of the present invention containing ROXY are different from other oxyfluorfen resistant rice lines in that the oxyfluorfen resistance trait is non-transgenic. As described in Examples 6 and 7 above, mutant allele ROXY is inherited as a single recessive gene. The PPO gene (also called PROTOX) has been identified in a rice transgenic study to provide resistance to PROTOX-inhibiting herbicides like oxyfluorfen (Jung, H. I. & Kuk, Y. I. J. Plant Biol. (2007) 50: 586. doi:10.1007/BF03030713). The rice PROTOX gene is located in the short arm of chromosome 1, and using the sequence of the reference genome, Nipponbare, a sequence of the region spanning the PROTOX gene was obtained from a public database. Sequencing of the PROTOX gene of the rice mutant lines 14G1 to 14G9 containing mutant allele ROXY and M-206 without mutant allele ROXY was performed in order to determine if there are any differences in the PROTOX sequences of the parent line M-206 and oxyfluorfen-resistant mutant lines, and to confirm if the source of resistance is indeed a mutated PROTOX gene. Leaf tissues were collected from lines 14G1, 2, 3, 4, 6, 7, 8, and 9 and M-206. DNA Extraction and Purification were done using Qiagen Plant Maxi Prep (column purification) at the Rice Experiment Station's DNA Marker Lab. Sequencing of the 4.5 kb region of rice Chromosome 1 containing the candidate gene, PROTOX, was performed by the Arizona Genome Institute, Tucson, Ariz. using the Nipponbare sequence as a standard japonica rice. The sequencing results showed that the PROTOX sequences of 14G1, 14G2, 14G3, 14G4, 14G6, 14G7, 14G8 and 14G9, containing mutant allele ROXY and M-206 without ROXY were identical, indicating that a mutation somewhere else in the rice genome is the cause of the resistance to oxyfluorfen.

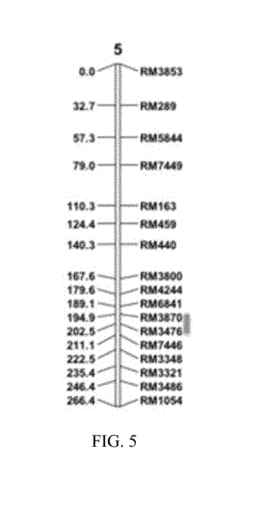

To determine the chromosomal location of mutant allele ROXY, a mapping population from the cross A-202×14G7 (see Example 6) was analyzed. Microsatellite markers were surveyed across the 12 chromosomes of rice. Only 98 out of 512 were polymorphic (19.1%) between the parents A-202 and 14G7. Polymorphic markers were used to examine the 166 mapping individuals. Regression analysis using the Qgene program (J. C. Nelson and R. Joehanes, 2010) revealed that the mutant allele ROXY is highly associated with RM3476 in Chromosome 5 at both 5% and 1% level of significance. Further analysis using composite interval analyses defined the ROXY region between markers RM3870 and RM3476 about 7.6 centimorgans. FIG. 5 shows that the location of mutant allele ROXY (orange mark) is flanked by markers RM3870 and RM3476 of Chromosome 5. Table 11 below shows the primer base sequences for the flanking markers RM3870 (Forward Sequence is SEQ ID NO:1; Reverse Sequence is SEQ ID NO:2) and RM3476 (Forward Sequence is SEQ ID NO:3; Reverse Sequence is SEQ ID NO:4). The sequence information was obtained from the Gramene Database.

TABLE 11

| Marker | | |
|---|---|---|
| Name | Forward Sequence | Reverse Sequence |
| RM3870 | TACATCTCCG | CCAAGGTTGA |
| | GCGTTTACAC | AACAGGAAGC |
| RM3476 | GATTCTCGTC | ATCCACGGTT |
| | GTAATCAAGA | AAGATAAATG |

In the unlikely event that another rice plant was found that had similar resistance to oxyfluorfen, one of ordinary skill in the art can perform marker analysis to determine whether the other rice plant contains mutant allele ROXY, located between markers RM3870 and RM3476 of rice Chromosome 5 using the above primer sequences. Additionally, one of ordinary skill in the art would know whether the other rice plant contained mutant allele ROXY by performing a complementation analysis, which can distinguish between allelic mutations in the same gene or in different genes. Complementation occurs when two strains of an organism with different homozygous recessive mutations that produce the same mutant phenotype, such as oxyfluorfen resistance, produce offspring with the wild-type phenotype when crossed. Since complementation will occur only if the mutations are in different genes, one skilled in the art will know whether the other rice line contains ROXY based on the results of the cross—if the mutations are the same, the mutant phenotype will occur, whereas if the mutations are different, the wild-type phenotype will occur.

This invention is directed to any rice seed or plant containing mutant allele ROXY. This invention also is directed to methods for producing a rice plant by crossing a first parent rice plant with a second parent rice plant wherein either the first or second parent rice plant is a rice plant containing mutant allele ROXY. Further, both first and second parent rice plants can comprise mutant allele ROXY. Still further, this invention also is directed to methods for producing a rice cultivar containing mutant allele ROXY by crossing a rice cultivar containing mutant allele ROXY with a second rice plant and growing the progeny seed, and repeating the crossing and growing steps with the rice cultivar containing mutant allele ROXY from 0 to 7 times. Thus, any such methods using mutant allele ROXY are part of this invention: selfing, backcrosses, hybrid production, crosses to populations, and the like. All plants produced using rice plants containing mutant allele ROXY as parents are within the scope of this invention, including plants derived from rice containing mutant allele ROXY. Advantageously, the rice line is used in crosses with other, different, rice lines to produce first generation (F₁) rice seeds and plants with superior characteristics.

It should be understood that rice plants containing mutant allele ROXY, the oxyfluorfen resistance trait, can, through routine manipulation of cytoplasmic or other factors, be produced in a male-sterile form. Such embodiments are also contemplated within the scope of the present claims.

As used herein, the term plant includes plant cells, plant protoplasts, plant cell tissue cultures from which rice plants can be regenerated, plant calli, plant clumps and plant cells that are intact in plants or parts of plants, such as embryos, pollen, ovules, flowers, glumes, panicles, leaves, stems, roots, root tips, anthers, pistils and the like.

The use of the terms “a,” “an,” and “the,” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. For example, if the range 10-15 is disclosed, then 11, 12, 13, and 14 are also disclosed. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

### DEPOSIT INFORMATION

A deposit of the California Cooperative Rice Research Foundation, Inc. proprietary rice seed containing mutant allele ROXY of the present invention disclosed above and recited in the appended claims has been made with the American Type Culture Collection (ATCC), 10801 University Boulevard, Manassas, Va. 20110 under the terms of the Budapest Treaty. The date of deposit was Aug. 25, 2016. The deposit of 2,500 seeds was taken from the same deposit maintained by California Cooperative Rice Research Foundation, Inc. since prior to the filing date of this application. All restrictions will be irrevocably removed upon granting of a patent, and the deposit is intended to meet all of the requirements of 37 C.F.R. § § 1.801-1.809. The ATCC Accession Number is PTA-123525. The deposit will be maintained in the depository for a period of thirty years, or five years after the last request, or for the enforceable life of the patent, whichever is longer, and will be replaced as necessary during that period.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

1. A rice plant having non-transgenic resistance to the herbicide oxyfluorfen.

2. A rice plant, a plant part thereof, or a rice seed having non-transgenic resistance to the herbicide oxyfluorfen, wherein said non-transgenic resistance to oxyfluorfen is conferred by mutant allele ROXY.

3. A rice plant, a plant part thereof, or a rice seed having non-transgenic resistance to the herbicide oxyfluorfen conferred by mutant allele ROXY, wherein a representative sample of seed containing said mutant allele ROXY was deposited under ATCC Accession No. PTA-123525.

4. A tissue culture of cells or protoplasts produced from the plant of claim 3, wherein said cells or protoplasts of the tissue culture are produced from a plant part selected from the group consisting of leaf, pollen, embryo, cotyledon, hypocotyl, meristematic cell, root, root tip, pistil, anther, flower, stem, glume and panicle.

5. A rice plant regenerated from the tissue culture of claim 4, wherein the plant contains mutant allele ROXY.

6. A method for producing an F₁ hybrid rice seed containing mutant allele ROXY, wherein the method comprises crossing the plant of claim 3 with a different rice plant and harvesting the resultant F₁ hybrid rice seed.

7. A hybrid rice seed produced by the method of claim 6.

8. A hybrid rice plant, or a plant part thereof, produced by growing said hybrid seed of claim 7, wherein said plant or plant part thereof contains mutant allele ROXY.

9. A method of producing an herbicide resistant rice plant wherein the method comprises transforming the rice plant of claim 3 with a transgene wherein the transgene confers resistance to an herbicide selected from the group consisting of imidazolinone, sulfonylurea, glyphosate, glufosinate, L-phosphinothricin, triazine, benzonitrile and acetyl CoA carboxylase inhibitors.

10. An herbicide resistant rice plant produced by the method of claim 9.

11. A method of producing an insect resistant rice plant wherein the method comprises transforming the rice plant of claim 3 with a transgene that confers insect resistance.

12. An insect resistant rice plant produced by the method of claim 11.

13. The rice plant of claim 12, wherein the transgene encodes a Bacillus thuringiensis endotoxin.

14. A method of producing a disease resistant rice plant wherein the method comprises transforming the rice plant of claim 3 with a transgene that confers disease resistance.

15. A disease resistant rice plant produced by the method of claim 14.

16. A method of producing a rice plant with modified fatty acid metabolism or modified carbohydrate metabolism wherein the method comprises transforming the rice plant of claim 3 with a transgene encoding a protein selected from the group consisting of fructosyltransferase, levansucrase, alpha-amylase, invertase and starch branching enzyme or DNA encoding an antisense of stearyl-ACP desaturase.

17. A rice plant having modified fatty acid metabolism or modified carbohydrate metabolism produced by the method of claim 16.

18. A method of producing a genetically modified rice plant, wherein the method comprises genome editing of the plant of claim 3.

19. A genetically modified rice plant produced by the method of claim 18.

20. A method of introducing a desired trait into a rice plant having non-transgenic resistance to the herbicide oxyfluorfen conferred by mutant allele ROXY wherein the method comprises:
(a) crossing a rice plant having non-transgenic resistance to the herbicide oxyfluorfen conferred by mutant allele ROXY, wherein a representative sample of seed containing mutant allele ROXY was deposited under ATCC Accession No. PTA-123525, with a plant of another rice cultivar that comprises a desired trait to produce progeny plants wherein the desired trait is selected from the group consisting of male sterility, herbicide resistance, insect resistance, modified fatty acid metabolism, modified carbohydrate metabolism and resistance to bacterial disease, fungal disease or viral disease;
(b) selecting one or more progeny plants that have the desired trait to produce selected progeny plants;
(c) backcrossing the selected progeny plants with the rice plant containing mutant allele ROXY to produce backcross progeny plants;
(d) selecting for backcross progeny plants that have the desired trait and contain mutant allele ROXY; and
(e) repeating steps (c) and (d) two or more times to produce selected third or higher backcross progeny plants that comprise the desired trait.

21. A plant produced by the method of claim 20, wherein the plant has the desired trait and contains mutant allele ROXY.

22. The plant of claim 21, wherein the desired trait is herbicide resistance and the resistance is conferred to an herbicide selected from the group consisting of imidazolinone, sulfonylurea, glyphosate, glufosinate, L-phosphinothricin, triazine, benzonitrile and acetyl CoA carboxylase inhibitors.

23. The plant of claim 21, wherein the desired trait is insect resistance and the insect resistance is conferred by a transgene encoding a Bacillus thuringiensis endotoxin.

24. The plant of claim 21, wherein the desired trait is modified fatty acid metabolism or modified carbohydrate metabolism and said desired trait is conferred by a nucleic acid encoding a protein selected from the group consisting of fructosyltransferase, levansucrase, alpha-amylase, invertase and starch branching enzyme or DNA encoding an antisense of stearyl-ACP desaturase.

25. A method of transferring mutant allele ROXY to a different genetic background, wherein the method comprises:
(a) obtaining the F₁ plant of claim 8;
(b) backcrossing said F₁ plant to a recipient parent plant not having mutant allele ROXY to produce backcross progeny plants;
(c) selecting for backcross progeny plants that contain mutant allele ROXY;
(d) backcrossing said selected backcross progeny plants to said recipient parent;
(e) repeating steps (c) and (d) two or more times in succession to produce selected third or higher backcross progeny plants that contain mutant allele ROXY; and harvesting the resultant seed.

26. A plant produced from the seed of claim 25, wherein said plant contains mutant allele ROXY.

27. A method for controlling or suppressing weeds at a rice production site, the method comprising the steps of:
(a) providing, at said site, a rice plant, plant part thereof, or rice seed of claim 2 containing mutant allele ROXY; and
(b) applying to said site an amount of herbicide effective to control or suppress weeds.

28. A method for growing a plant of claim 2 while controlling or suppressing weeds in the vicinity of said plant, said method comprising the steps of:
(a) growing said plant; and
(b) applying herbicide to said plant and weeds at a level of herbicide that would inhibit the growth of a corresponding rice plant not containing mutant allele ROXY.

29. A method for controlling or suppressing weeds at a rice production site, the method comprising the steps of:
(a) applying to said site an amount of herbicide effective to control or suppress weeds; and
(b) providing, at said site, the rice plant, plant part thereof, or rice seed of claim 2 containing mutant allele ROXY.

30. A method for growing a plant of claim 2 while controlling or suppressing weeds in the vicinity of said plant, said method comprising the steps of:
(a) applying herbicide in the vicinity where said plant will grow, at a level of herbicide that would inhibit the growth of a corresponding rice plant not containing mutant allele ROXY; and
(b) growing said plant.

31. The rice plant, plant part thereof, or rice seed of claim 2, wherein said rice plant, plant part thereof, or rice seed has resistance to PPO inhibiting herbicides, herbicide mixtures or herbicide combinations with oxyfluorfen.

32. The rice plant, plant part thereof, or rice seed of claim 31, wherein said herbicide mixtures or herbicides used in combination with oxyfluorfen are selected from the group consisting of imidazolinone, sulfonylurea, glyphosate, glufosinate, L-phosphinothricin, triazine, benzonitrile and acetyl CoA carboxylase inhibitors.
